SEQUENCE LISTING (20 sequences)

SEQ ID NO: 1 - DNA, 20 bp, Artificial Sequence
  Akr1B3 primer forward
ggccgtgaaagttgctattg

SEQ ID NO: 2 - DNA, 21 bp, Artificial Sequence
  Akr1B3 primer reverse
atgctcttgtcatggaacgtg

SEQ ID NO: 3 - DNA, 21 bp, Artificial Sequence
  Cox-1 primer forward
atgagtcgaaggagtctctcg

SEQ ID NO: 4 - DNA, 22 bp, Artificial Sequence
  Cox-1 primer reverse
gcacggatagtaacaacaggga

SEQ ID NO: 5 - DNA, 19 bp, Artificial Sequence
  Cox-2 primer forward
aagccgagcacctttggag

SEQ ID NO: 6 - DNA, 23 bp, Artificial Sequence
  Cox-2 primer reverse
attgatggtggctgttttggtag

SEQ ID NO: 7 - DNA, 19 bp, Artificial Sequence
  mpges-1 primer forward
ctagccgagatgccttccc

SEQ ID NO: 8 - DNA, 20 bp, Artificial Sequence
  mpges-1 primer reverse
ccaccgcgtacatcttgatg

SEQ ID NO: 9 - DNA, 20 bp, Artificial Sequence
  mpges-2 primer forward
tccttgccctggtcattcat

SEQ ID NO: 10 - DNA, 20 bp, Artificial Sequence
  mpges-2 primer reverse
ggaaggagacagcttgcaac

SEQ ID NO: 11 - DNA, 20 bp, Artificial Sequence
  cpges primer forward
agttgtcttggaggaagcga

SEQ ID NO: 12 - DNA, 19 bp, Artificial Sequence
  cpges primer reverse
atgactggccggattctcc

SEQ ID NO: 13 - DNA, 20 bp, Artificial Sequence
  Txs primer forward
cacaaacacgctgtccttca

SEQ ID NO: 14 - DNA, 20 bp, Artificial Sequence
  Txs primer reverse
ttccccatgaagaggtccac

SEQ ID NO: 15 - DNA, 19 bp, Artificial Sequence
  H-pgds primer forward
tgggaagacagcgttggag

SEQ ID NO: 16 - DNA, 19 bp, Artificial Sequence
  H-pgds primer reverse
aggcgaggtgcttgatgtg

SEQ ID NO: 17 - DNA, 23 bp, Artificial Sequence
  L-pgds primer forward
cctcaatctcacctctaccttcc

SEQ ID NO: 18 - DNA, 20 bp, Artificial Sequence
  L-pgds primer reverse
tcatagttggcctccaccac

SEQ ID NO: 19 - DNA, 22 bp, Artificial Sequence
  18s-rRNA primer forward
gactcaacacgggaaacctcac

SEQ ID NO: 20 - DNA, 20 bp, Artificial Sequence
  18s-rRNA primer reverse
cacccacggaatcgagaaag

(19) United States
(12) Patent Application Publication
(10) Pub. No.: US 2023/0160914 A1
(43) Pub. Date: May 25, 2023

(54) Method for Detecting Atopic Dermatitis

(71) Applicants: The University of Tokyo, Tokyo (JP); NATIONAL CENTER FOR CHILD HEALTH AND DEVELOPMENT, Tokyo (JP)

(72) Inventors: Takahisa Murata, Tokyo (JP); Tatsuro Nakamura, Tokyo (JP); Yuta Hamasaki, Tokyo (JP); Yukihiro Ohya, Tokyo (JP); Yusuke Inuzuka, Tokyo (JP); Kiwako Yamamoto, Tokyo (JP)

(21) Appl. No.: 17/766,118

(22) PCT Filed: Oct. 2, 2020
(86) PCT No.: PCT/JP2020/037645

§ 371 Date: Apr. 1, 2022

(30) Foreign Application Priority Data
Oct. 4, 2019 (JP) 2019-184174

(51) Int. Cl. G01N 33/92; C12Q 1/6883

(52) U.S. Cl. CPC G01N 33/92; C12Q 1/6883; C12Q 2600/158; C12Q 1/6851

(57) ABSTRACT

An object of the present disclosure is to provide a novel method for detecting atopic dermatitis and a novel method for determining a therapeutic effect on atopic dermatitis. The present disclosure provides a method for detecting atopic dermatitis, including the step of measuring the concentration of a lipid metabolite in a biological sample of a subject. The present disclosure also provides a method for determining a therapeutic effect on atopic dermatitis, including the step of measuring the concentration of a lipid metabolite in a biological sample of a subject.

### CROSS-REFERENCE TO RELATED APPLICATION

This application is the United States national phase of International Application No. PCT/JP2020/037645 filed Oct. 2, 2020, and claims priority to Japanese Patent Application No. 2019-184174 filed Oct. 4, 2019, the disclosures of which are hereby incorporated by reference in their entirety.

### SEQUENCE LISTING

The Sequent Listing associated with this application is filed in electronic format via EFS-Web and is hereby incorporated by reference into the specification in its entirety. The name of the text file containing the Sequence Listing is 08577-2202073_ST25.txt. The size of the text file is 500 bytes, and the text file was created on Aug. 1, 2022.

### BACKGROUND OF THE INVENTION

### Field of the Invention

The present invention relates to a method for detecting atopic dermatitis. The present invention also relates to a method for determining a therapeutic effect on atopic dermatitis.

### Description of Related Art

Atopic dermatitis (AD) is an inflammatory skin disease characterized by pruritic eczema, which repeatedly exacerbates and remits. AD is caused by the interaction of genetic and environmental factors, and the number of AD patients has increased in recent years. The prevalences of children and adults are estimated to be 9.8 to 13.2% and 2.5 to 9.4%, respectively. It has also been reported that the onset of AD in infancy increases the risk of developing other allergic conditions such as food allergies, bronchial asthma and allergic rhinitis. In AD lesions, Th2 cells correlate with Th2 cytokines such as IL-4 and IL-13 and chemokines such as TARC, and the serum TARC level is known to be useful as a marker for AD (Non-Patent Documents 1 and 2).
Non-Patent Document 1: Thijs J et al., Current Opinion in Allergy and Clinical Immunology, 15(5):453-460 (2015)
Non-Patent Document 2: Judith L et al., J. Clin. Med, 4, 479-487(2015)

### SUMMARY OF THE INVENTION

An object of the present disclosure is to provide a novel method for detecting atopic dermatitis. Another object of the present disclosure is to provide a novel method for determining a therapeutic effect on atopic dermatitis.

The present inventors have confirmed that the amount of a lipid metabolite in biological samples of atopic dermatitis model mice and atopic dermatitis patients is different from that of the lipid metabolite in biological samples of healthy subjects. Further, the present inventors have found that the concentration of the lipid metabolite can be used as an index to detect atopic dermatitis. The present disclosure is based on these findings.

According to the present disclosure, there are provided embodiments set forth in the following clauses:

Clause 1. A method for detecting, or a method for diagnosing, atopic dermatitis, comprising the step of measuring the concentration of a lipid metabolite in a biological sample of a subject.

Clause 2. The method for detection or the method for diagnosis according to clause 1, which further comprises the step of comparing the concentration of the lipid metabolite in the biological sample of the subject with the concentration of the lipid metabolite in a biological sample of a healthy subject.

Clause 3. The method for detection or the method for diagnosis according to clause 1 or clause 2, wherein, when the concentration of the lipid metabolite in the biological sample of the subject is significantly different from the concentration of the lipid metabolite in the biological sample of the healthy subject, it is indicated that the subject suffers from atopic dermatitis.

Clause 4. The method for detection or the method for diagnosis according to any one of clauses 1 to 3, wherein the biological sample is a body fluid.

Clause 5. A method for determining a therapeutic effect on atopic dermatitis, comprising the step of measuring the concentration of a lipid metabolite in a biological sample of a subject.

Clause 6. The method for determination according to clause 5, which further comprises the step of comparing the concentration of the lipid metabolite in the biological sample of the subject with the concentration of the lipid metabolite in a biological sample of a healthy subject.

Clause 7. The method for determination according to clause 5 or clause 6, wherein, when the concentration of the lipid metabolite in the biological sample of the subject is significantly different from the concentration of the lipid metabolite in the biological sample of the subject before treatment or the concentration of the lipid metabolite in a biological sample of a subject suffering from atopic dermatitis, or when the concentration of the lipid metabolite in the biological sample of the subject is not significantly different from the concentration of the lipid metabolite in the biological sample of the healthy subject, it is indicated that the treatment has a therapeutic effect.

Clause 8. The method for determination according to any one of clauses 5 to 7, wherein treatment of atopic dermatitis is drug therapy or proactive therapy.

Clause 9. The method for determination according to any one of clauses 5 to 8, wherein the biological sample is a body fluid.

Clause 10. The method for detection or the method for diagnosis according to any one of clauses 1 to 4, or the method for determination according to any one of clauses 5 to 9, wherein the lipid metabolite is one or more lipid metabolite(s) selected from the group consisting of an arachidonic acid metabolite, an eicosapentaenoic acid metabolite, and a docosahexaenoic acid metabolite.

Clause 11. The method for detection or the method for diagnosis according to any one of clauses 1 to 4 and 10, or the method for determination according to any one of clauses 5 to 10, wherein the lipid metabolite tends to have a higher concentration in the biological sample of the subject suffering from atopic dermatitis than in the biological sample of the healthy.

Clause 12. The method for detection or the method for diagnosis according to clause 11, or the method for determination according to clause 11, wherein the lipid metabolite is one or more lipid metabolite(s) selected from the group consisting of 13,14-dihydro-15-keto-PGJ2, tetranor-PGDM, 20-hydroxy-PGE2, 15-keto-PGE2, 13,14-dihydro-15-keto-tetranor-PGE2, tetranor-PGEM, 15-keto-PGF2α, 13,14-dihydro-15-keto-tetranor-PGF1α, tetranor-PGFM, 6,15-diketo-13,14-dihydro-PGF1α, 5-HpETE, 17-HETE, 11β-13,14-dihydro-15-keto-PGF2α, PGK2, 13,14-dihydro-15-keto-tetranor-PGF1β, 13,14-dihydro-15-keto-PGE2, 6-keto-PGF1α, and TXB2.

Clause 13. The method for detection or the method for diagnosis according to any one of clauses 1 to 4 and 10, or the method for determination according to any one of clauses 5 to 10, wherein the lipid metabolite tends to have a lower concentration in the biological sample of the subject suffering from atopic dermatitis than in the biological sample of the healthy.

Clause 14. The method for detection or the method for diagnosis according to clause 13, or the method for determination according to clause 13, wherein the lipid metabolite is one or more lipid metabolite(s) selected from the group consisting of 5-HETE, arachidonic acid (AA), 11β-13,14-dihydro-15-keto-PGF2α, iPF2α-IV, EPA, 4-HDoHE, 10,17-DiHDoHE, and PGD3.

Clause 15. The method for detection or the method for diagnosis according to any one of clauses 1 to 4 and 10-14, or the method for determination according to any one of clauses 5 to 14, wherein the concentration of the lipid metabolite is measured by mass spectrometry.

Clause 16. An atopic dermatitis marker, comprising a lipid metabolite.

Clause 17. The atopic dermatitis marker according to clause 16, wherein the lipid metabolite is one or more lipid metabolite(s) selected from the group consisting of an arachidonic acid metabolite, an eicosapentaenoic acid metabolite, and a docosahexaenoic acid metabolite.

Clause 18. The atopic dermatitis marker according to clause 16 or clause 17, wherein the lipid metabolite is one or more lipid metabolite(s) selected from the group consisting of 13,14-dihydro-15-keto-PGJ2, tetranor-PGDM, 20-hydroxy-PGE2, 15-keto-PGE2, 13,14-dihydro-15-keto-tetranor-PGE2, tetranor-PGEM, 15-keto-PGF2α, 13,14-dihydro-15-keto-tetranor-PGF1α, tetranor-PGFM, 6,15-diketo-13,14-dihydro-PGF1α, 5-HpETE, 17-HETE, 11β-13,14-dihydro-15-keto-PGF2α, PGK2, 13,14-dihydro-15-keto-tetranor-PGF1β, 13,14-dihydro-15-keto-PGE2, 6-keto-PGF1α, TXB2, 5-HETE, arachidonic acid (AA), iPF2α-IV, EPA, 4-HDoHE, 10,17-DiHDoHE, and PGD3.

Clause 19. A method for screening for a therapeutic agent or relieving agent for atopic dermatitis, comprising the steps of administering a candidate for a therapeutic agent or relieving agent for atopic dermatitis to a subject; and measuring the concentration of a lipid metabolite in a biological sample of the subject.

Clause 20. The method for screening according to clause 19, which further comprises the step of comparing the concentration of the lipid metabolite in the biological sample of the subject after administration of the candidate for the therapeutic agent or relieving agent with the concentration of the lipid metabolite in a biological sample of a healthy subject.

Clause 21. The method for screening according to clause 19 or clause 20, wherein, when the concentration of the lipid metabolite in the biological sample of the subject after administration of the candidate for the therapeutic agent or relieving agent is significantly different from the concentration of the lipid metabolite in the biological sample of the subject before administration of the candidate for the therapeutic agent or relieving agent or the concentration of the lipid metabolite in a biological sample of a subject suffering from atopic dermatitis, or when the concentration of the lipid metabolite in the biological sample of the subject is not significantly different from the concentration of the lipid metabolite in the biological sample of the healthy subject, it is indicated that the candidate for the therapeutic agent or relieving agent has a therapeutic effect.

Clause 22. A method for identifying an atopic dermatitis marker in a lipid metabolite in a biological sample, comprising the steps of measuring the concentration of a lipid metabolite in a biological sample of a subject suffering from atopic dermatitis and the concentration of the lipid metabolite in a biological sample of a healthy subject; and comparing the measured two concentrations.

Clause 23. The method for identification according to clause 22, wherein, when the concentration of the lipid metabolite in the biological sample of the subject suffering from atopic dermatitis is significantly different from the concentration of the lipid metabolite in the biological sample of the healthy subject, it is indicated that the lipid metabolite is an atopic dermatitis marker.

Clause 24. A kit for detecting, or a kit for diagnosing, atopic dermatitis, comprising a means for quantifying a lipid metabolite in a biological sample of a subject.

Clause 25. A method for treating atopic dermatitis, comprising the step (A) of measuring the concentration of a lipid metabolite in a biological sample of a subject; the step (B) of comparing the concentration of the lipid metabolite in the biological sample of the subject with the concentration of the lipid metabolite in a biological sample of a healthy subject; the step (C) of, when the concentration of the lipid metabolite in the biological sample of the subject is significantly different from the concentration of the lipid metabolite in the biological sample of the healthy subject, determining that the subject suffers from atopic dermatitis; and the step (N) of performing treatment of atopic dermatitis on a subject determined to suffer from atopic dermatitis.

The present disclosure is advantageous in enabling quantitative detection of atopic dermatitis and a therapeutic effect on atopic dermatitis based on a biological sample of a test subject. The present disclosure is also advantageous in that a non-invasively collected biological sample can be used as the biological sample of the test subject.

### BRIEF DESCRIPTION OF THE DRAWINGS

FIG. 1 shows procedures for preparing atopic dermatitis model mice on a time-series basis.

FIG. 2 shows typical photographs of the back skin of an AD model mouse on the DNFB pretreatment, first, second, and third DNFB stimulations.

FIG. 3 shows dermatitis scores of the AD model mice on the DNFB pretreatment, the first, second, and third DNFB stimulations. The data is indicated as mean±standard error (n=8). **P<0.01 indicates a significant difference when the score is compared with that of the DNFB pretreatment.

FIG. 4 shows numbers of times of scratching of the AD model mice on the DNFB pretreatment, the first, second, and third DNFB stimulations. The data is indicated as mean±standard error (n=8). *P<0.05 and **P<0.01 each indicate a significant difference when the number is compared with that of the DNFB pretreatment. ^{†}P<0.05 and ^{††}P<0.01 each indicate a significant difference when the number is compared with that of the first DNFB stimulation.

FIG. 5 shows ear thicknesses of the AD model mice on the DNFB pretreatment, the first, second, and third stimulations. **P<0.01 indicates a significant difference when the ear thickness is compared with that of the DNFB pretreatment.

FIG. 6 shows transepidermal water losses (TEWL) on the back of the AD model mice on the DNFB pretreatment, the first, second, and third DNFB stimulations. *P<0.05 and **P<0.01 each indicate a significant difference when the TEWL is compared with that of the DNFB pretreatment.

FIG. 7 shows typical examples of HE-stained images of a back skin lesion fragment of an AD model mouse on the DNFB pretreatment, the first, second, and third DNFB stimulations. The scale bar indicates 50 μm.

FIG. 8 shows epidermal thickness of the AD model mice measured from the HE-stained images. The data is indicated as mean±standard error (n=5 to 7). **P<0.01 indicates a significant difference when the epidermal thickness is compared with the DNFB pretreatment. ^{†}P<0.05 indicates a significant difference when the epidermal thickness is compared with that of the first DNFB stimulation.

FIGS. 9A and 9B show typical examples of CAE-stained images of the AD model mice (A: mast cells, B: neutrophils).

FIGS. 10A and 10B show numbers of mast cells (A) and numbers of neutrophils (B) in the AD model mice, which were quantified from the CAE-stained images on the DNFB treatment, the first and third DNFB stimulations. The data is indicated as mean±standard error (n=5 to 7). **P<0.01 indicates a significant difference when the number is compared with that of the DNFB pretreatment.

FIG. 11 shows typical examples of MGG-stained images of the AD model mice (eosinophils).

FIG. 12 shows numbers of eosinophils in the AD model mice, which were quantified from the MGG-stained images on the DNFB pretreatment, the first and third DNFB stimulations. The data is indicated as mean±standard error (n=5 to 7). **P<0.01 indicates a significant difference when the number is compared with that of the DNFB pretreatment.

FIGS. 13A to 13C show amounts of lipid mediators at the downstream of COX derived from AA and metabolized via PGD2, which were excreted in urine of the AD model mice. *P<0.05 and **P<0.01 each indicate a significant difference when the amount is compared with that of the DNFB pretreatment.

FIGS. 14A to 14E show amounts of lipid mediators at the downstream of COX derived from AA and metabolized via PGE2, which were excreted in urine of the AD model mice. *P<0.05 and **P<0.01 each indicate a significant difference when the amount is compared with that of the DNFB pretreatment. ^{††}P<0.01 indicates a significant difference when the amount is compared with that of the first DNFB stimulation.

FIG. 15 shows an amount of a lipid mediator at the downstream of COX derived from AA and metabolized via PGF2α, which was excreted in urine of the AD model mice. **P<0.01 indicates a significant difference when the amount is compared with that of the DNFB pretreatment. ^{†}P<0.05 indicates a significant difference when the amount is compared with that of the first DNFB stimulation.

FIG. 16 shows an amount of a lipid mediator at the downstream of COX derived from AA and metabolized via PGI2, which was excreted in urine of the AD model mice. *P<0.05 indicates a significant difference when the amount is compared with that of the DNFB pretreatment. ^{†}P<0.05 indicates a significant difference when the amount is compared with that of the first DNFB stimulation.

FIG. 17 shows amounts of TXB2 at the downstream of COX derived from AA, which was excreted in urine of the AD model mice. **P<0.01 indicates a significant difference when the amount is compared with that of the DNFB pretreatment.

FIGS. 18A and 18B show amounts of lipid mediators derived from AA and produced by enzyme-independent oxidation, which were excreted in urine of the AD model mice. *P<0.05 indicates a significant difference when the amount is compared with that of the DNFB pretreatment. ^{†}P<0.05 indicates a significant difference when the amount is compared with that of the first DNFB stimulation.

FIGS. 19A and 19B show amounts of lipid mediators at the downstream of LOX derived from n-3 fatty acids, which were excreted in urine of the AD model mice. *P<0.05 and **P<0.01 each indicate a significant difference when the amount is compared with that of the DNFB pretreatment. ^{†}P<0.05 and ^{††}P<0.01 each indicate a significant difference when the amount is compared with that of the first DNFB stimulation.

FIGS. 20A to 20I show mRNA expression levels of cox-1 (A), cox-2 (B), mpges-1 (C), Akr1b3 (D), Txs (E), H-pgds (F), mpges-2 (G), cpges (H) and L-pgds (I) extracted from skin lesions of the AD model mice after the third DNFB stimulation. The data is indicated as mean±standard error (n=5 to 7, for each). *P<0.05 and **P<0.01 each indicate a significant difference when the mRNA expression level is compared with that of a control (solvent-treated).

FIG. 21 shows typical examples of immuno-stained images of COX-1, COX-2, mPGES-1, AKR1B3, solvent (vehicle), normal goat serum, normal rabbit IgG, and TXS in the skin lesions of the AD model mice. The scale bar indicates 50 μm.

FIGS. 22A and 22B show amounts of lipid mediators at the downstream of COX (derived from AA and metabolized via PGD2), which were excreted in urine of AD patients (n=10 to 14). The data is a ratio relative to an internal standard, and is expressed as mean value±standard error. *P<0.05 indicates a significant difference when the amount is compared with that of the control (n=3 to 4, for each).

FIGS. 23A to 23D show amounts of lipid mediators at the downstream of COX derived from AA and metabolized via PGE2, which were excreted in urine of the AD patients (n=10 to 14). The data is a ratio relative to an internal standard, and is expressed as mean value±standard error. *P<0.05 indicates a significant difference when the amount is compared with that of the control (n=3 to 4, for each).

FIGS. 24A to 24C show amounts of lipid mediators at the downstream of COX derived from AA and metabolized via PGF2α, which were excreted in urine of the AD patients (n=10 to 14). The data is a ratio relative to an internal standard, and is expressed as mean value±standard error. *P<0.05 indicates a significant difference when the amount is compared with that of the control (n=3 to 4).

FIG. 25 shows an amount of a lipid mediator at the downstream of COX derived from AA and metabolized via PGI2, which was excreted in urine of the AD patients. The data is a ratio relative to an internal standard, and is expressed as mean value±standard error. *P<0.05 indicates a significant difference when the amount is compared with that of the control (n=3 to 4).

FIGS. 26A and 26B show amounts of lipid mediators at the downstream of LOX derived from AA and metabolized via PGF2α, which were excreted in urine of the AD patients. The data is a ratio relative to an internal standard, and is expressed as mean value±standard error. *P<0.05 indicates a significant difference when the amount is compared with that of the control (n=3 to 4).

FIG. 27 shows an amount of a lipid mediator at the downstream of CYP derived from AA and metabolized via PGF2α, which was excreted in urine of the AD patients. The data is a ratio relative to an internal standard, and is expressed as mean value±standard error. *P<0.05 indicates a significant difference when the amount is compared with that of the control (n=3 to 4).

FIGS. 28A and 28B show amounts of AA and a lipid mediator produced by enzyme-independent oxidation of AA, which were excreted in urine of the AD patients. The data is a ratio relative to an internal standard, and is expressed as mean value±standard error. *P<0.05 and **P<0.01 each indicate a significant difference when the amount is compared with that of the control (n=3 to 4).

FIG. 29 shows an amount of EPA which was excreted in urine of the AD patients (n=10 to 14). The data is a ratio relative to an internal standard, and is expressed as mean value±standard error. *P<0.05 indicates a significant difference when the amount is compared with that of the control (n=3 to 4).

FIGS. 30A and 30B show amounts of lipid mediators at the downstream of LOX derived from DHA, which were excreted in urine of the AD patients (n=10 to 14). The data is a ratio relative to an internal standard, and is expressed as mean value±standard error. *P<0.05 indicates a significant difference when the amount is compared with that of the control (n=3 to 4).

FIG. 31A shows an experiment using mice subjected to tape stripping treatment (non-allergic dermatitis model mice) on a time-series basis. FIG. 31B shows typical examples of HE-stained images of a back skin fragment of a non-allergic dermatitis model mouse. The scale bar indicates 50 μm. FIG. 31C shows epidermal thickness of the non-allergic dermatitis model mice measured from the HE-stained images. The data is indicated as mean±standard error (n=4 to 7). FIG. 31D shows mRNA expression levels of Tslp, Il-4, Il-13 and Ccl17 extracted from the skin of the non-allergic dermatitis model mice. The data is indicated as mean±standard error (n=4 to 7). FIG. 31E shows amounts of lipid mediators at the downstream of COX derived from AA, which were excreted in urine of the non-allergic dermatitis model mice. The data is a ratio relative to an internal standard, and is expressed as mean±standard error (m=4 to 7). *P<0.05 indicates a significant difference when the amount is compared with that on the day when tape stripping treatment was performed (Day 0). FIG. 31F shows mRNA expression levels of Cox-2, mPGES-2, Akr1B3 and H-pgds extracted from the skin of the non-allergic dermatitis model mice. The data is indicated as mean±standard error (n=4 to 7). FIG. 31G shows typical examples of immuno-stained images of the skin of the non-allergic dermatitis model mice (left: ISO (negative control with an isotype antibody), and right: AKR1B3 antibody). The scale bar indicates 50 μm.

FIG. 32A shows numbers of eosinophils (left), numbers of neutrophils (middle), and numbers of mast cells (right), which were quantified from MGG-stained images of the non-allergic dermatitis model mice. The data is indicated as mean±standard error (n=4 to 7). *P<0.05 and **P<0.01 each indicate a significant difference when the amount is compared with that of the control (n=4 to 7). FIG. 32B shows amounts of a lipid mediator metabolized via PGF2a (left) and lipid mediators metabolized via PGE2 (middle and right), which were excreted in urine of the non-allergic dermatitis model mice are shown.

### DETAILED DESCRIPTION OF THE INVENTION

In the present disclosure, the term “lipid metabolite” means a lipid degradation product produced in vivo by degradation caused by enzyme-dependent oxidation or enzyme-independent oxidation (sometimes abbreviated as “OX” herein), and includes lipid mediators having a physiological effect of controlling inflammatory reactions. Enzyme-dependent oxidation progresses by a lipid metabolic enzyme present in vivo. The enzyme includes lipid metabolic enzymes involved in onset and development of atopic dermatitis (preferably, lipid metabolic enzymes activated by the onset and development of atopic dermatitis), such as cyclooxygenase (sometimes abbreviated as “COX” herein), lipoxygenase (sometimes abbreviated as “LOX” herein) (for example, 5-LOX and 15-LOX), and cytochrome p450 (sometimes abbreviated as “CYP” herein). Examples of the lipid that is degraded by enzyme-dependent oxidation or enzyme-independent oxidation include arachidonic acid, eicosapentaenoic acid, and docosahexaenoic acid.

Examples of the lipid metabolite in the present disclosure include arachidonic acid metabolites, eicosapentaenoic acid metabolites, and docosahexaenoic acid metabolites. The lipid metabolites can be classified into lipid metabolites whose concentration in a biological sample of a subject suffering from atopic dermatitis tends to be higher than (or beyond) the concentration thereof in a biological sample of a healthy subject (lipid metabolite X) and lipid metabolites whose concentration in the biological sample of the subject suffering from atopic dermatitis tends to be lower than (or below) the concentration thereof in the biological sample of the healthy subject (lipid metabolite Y). Examples of the lipid metabolite X include COX metabolites of arachidonic acid (e.g., 11β-13,14-dihydro-15-keto-PGF2α, 13,14-dihydro-15-keto-PGJ2, tetranor-PGDM (tetranor-Prostaglandin D Metabolite) (as used herein, the “tetranor-PGDM” is 9-hydroxy-11,15-dioxo-13,14-dihydro-2,3,4,5-tetranor-prostan-1,20-dioic acid), 20-hydroxy-PGE2, 15-keto-PGE2, 13,14-dihydro-15-keto-tetranor-PGE2, tetranor-PGEM (tetranor-Prostaglandin E Metabolite) (as used herein, the “tetranor-PGEM” is 9,15-dioxo-11-ydroxy-13,14-dihydro-2,3,4,5-tetranor-prostan-1,20-dioic acid), 15-keto-PGF2α, 13,14-dihydro-15-keto-tetranor-PGF1α, tetranor-PGFM (tetranor-Prostaglandin F Metabolite) (as used herein, the “tetranor-PGFM” is 9α,11-dihydroxy-15-oxo-13,14-dihydro-2,3,4,5-tetranor-prostan-1,20-dioic acid), 6,15-diketo-13,14-dihydro-PGF1α, PGK2, 13,14-dihydro-15-keto-tetranor-PGF1β, 13,14-dihydro-15-keto-PGE2, 6-keto-PGF1α, and TXB2), LOX metabolites of arachidonic acid (e.g., 5-LOX metabolites of arachidonic acid such as 5-HpETE), CYP metabolites of arachidonic acid (e.g., 17-HETE), and OX metabolites of arachidonic acid (e.g., 8-iso-15(R)-PGF2α). Preferred examples of the lipid metabolite X include those characterized by having a significantly higher concentration in the biological sample of the subject suffering from atopic dermatitis than in the biological sample of the healthy subject. Examples of the lipid metabolite Y include AA, COX metabolites of arachidonic acid (e.g., 11β-13,14-dihydro-15-keto-PGF2α), LOX metabolites of arachidonic acid (e.g., 5-LOX metabolites of arachidonic acid such as 5-HETE), OX metabolites of arachidonic acid (e.g., iPF2α-IV), LOX metabolites of eicosapentaenoic acid and docosahexaenoic acid (e.g., 5-LOX metabolites of docosahexaenoic acid such as 4-HDoHE and 15-LOX metabolites of docosahexaenoic acid such as 10,17-DiHDoHE), and COX metabolites of eicosapentaenoic acid (e.g., COX metabolites of eicosapentaenoic acid such as PGD3). Preferred examples of the lipid metabolite Y include those characterized by having a significantly lower concentration in the biological sample of the subject suffering from atopic dermatitis than in the biological sample of the healthy subject.

In the present disclosure, the term “atopic dermatitis” means a disease involving pruritic eczema as the main lesion, which repeatedly exacerbates and remits, and most of patients have atopic diathesis (allergic constitution). It is an eczematous disease with a characteristic bilaterally symmetric distribution, which varies in favorite site depending on age, develops in infancy and remits in childhood or repeatedly recurs without remission, and chronically exhibits characteristic eczematous lesions whose symptoms persist into adulthood (2018 Guidelines for Management of Atopic Dermatitis).

In the present disclosure, the term “biological sample” means a sample separated from a living body, and represents, for example, a body fluid such as urine, blood, saliva, runny nose, sweat, tears, or feces. A method for collecting the biological sample may be invasive or noninvasive, and can be selected according to the test subject.

In the present disclosure, the term “subject” is used with the meaning including not only a human, but also a mammal other than a human (for example, monkeys, mice, rats, dogs, cats, rabbits, horses, cows, pigs and sheep).

According to a first aspect of the present disclosure, there is provided a method for detecting atopic dermatitis. The method for detection according to the present disclosure can be used to detect atopic dermatitis using a lipid metabolite in a biological sample as an index.

In the method for detection according to the present disclosure, first, a step (A) of measuring the concentration of a lipid metabolite in a biological sample of a test subject is carried out. The concentration of the lipid metabolite can be measured by a known method. For example, the concentration of the lipid metabolite can be measured by mass spectrometry, an ELISA method and an immunoassay such as an immunochromatography method. Examples of the mass spectrometry include liquid chromatography-mass spectrometry (LC-MS), liquid chromatography-tandem mass spectrometry (LC-MSMS), high performance liquid chromatography-mass spectrometry (HPLC-MS), and high performance liquid chromatography-tandem mass spectrometry (HPLC-MSMS). The immunoassay is an analytical method using a detectably-labeled anti-lipid metabolite antibody or a detectably-labeled antibody (secondary antibody) against an anti-lipid metabolite antibody. Depending on the antibody labeling method, the immunoassays are classified into enzyme immunoassay (EIA or ELISA), radioimmunoassay (RIA), fluorescence immunoassay (FIA), fluorescence polarization immunoassay (FPIA), chemiluminescence immunoassay (CLIA), and the like, all of which can be used in the present disclosure. From the viewpoint of accurately measuring the concentrations of lipid metabolites having similar structures, measurement by mass spectrometry (especially, LC-MSMS and HPLC-MSMS) is preferred.

In the method for detection of the present disclosure, the step of determining, in the subject from whom/which the biological sample has been collected, the presence or absence of atopic dermatitis based on the concentration of the lipid metabolite measured in the step (A) can further be carried out. This step may further comprise a step (B) of comparing the concentration of the lipid metabolite in the biological sample of the subject with the concentration of the lipid metabolite in a biological sample of a healthy subject. When the concentration of the lipid metabolite in the biological sample of the subject is significantly different from the concentration of the lipid metabolite in the biological sample of the healthy subject, it is indicated that the subject suffers from atopic dermatitis. That is, the method for detection of the present disclosure may further comprise a step (C) of determining that the subject suffers from atopic dermatitis when the concentration of the lipid metabolite in the biological sample of the subject is significantly different from the concentration of the lipid metabolite in the biological sample of the healthy subject. The term “significantly different” in the step (C) means that the concentration of the lipid metabolite in the subject is either higher or lower than the concentration of the lipid metabolite in the healthy subject depending on the type of lipid metabolite, for example, that, when the lipid metabolite is a lipid metabolite X, the concentration of the lipid metabolite in the biological sample of the subject is higher, preferably significantly higher, than the concentration of the lipid metabolite in the biological sample of the healthy subject (for example, the concentration of the lipid metabolite in the biological sample of the subject is statistically significantly high as compared with the concentration of the lipid metabolite in the biological sample of the healthy subject, or is about 1.1 times or more, about 1.2 times or more, about 1.3 times or more, about 1.4 times or more, about 1.5 times or more, about 1.6 times or more, about 1.7 times or more, about 1.8 times or more, about 1.9 times or more, about 2.0 times or more, about 2.5 times or more, or about 3 times or more thereof), and means that, when the lipid metabolite is a lipid metabolite Y, the concentration of the lipid metabolite in the biological sample of the subject is lower, preferably significantly lower, than the concentration of the lipid metabolite in the biological sample of the healthy subject (for example, the concentration of the lipid metabolite in the biological sample of the subject is statistically significantly low as compared with the concentration of the lipid metabolite in the biological sample of the healthy subject, or is about 0.9 times or less, about 0.8 times or less, about 0.7 times or less, about 0.6 times or less, about 0.5 times or less, about 0.4 times or less, or about 0.3 times or less thereof). As the concentration of the lipid metabolite in the biological sample of the healthy subject, there can be used a mean value calculated by collecting biological samples from a plurality of healthy subjects in advance and measuring the concentrations of the lipid metabolite in the biological samples. The phrase “suffers from atopic dermatitis” is used with the meaning including also the case where the subject may suffer from atopic dermatitis.

According to the method for detection of the present disclosure, atopic dermatitis can be detected in the test subject. Therefore, the method for detection of the present disclosure can be used as an auxiliary method in the diagnosis of atopic dermatitis, and, finally, a doctor or veterinarian can decide whether or not the subject suffers from atopic dermatitis, in combination with other findings in some cases.

The method for detection of the present disclosure can be used to quantitatively detect atopic dermatitis based on the biological sample collected from the subject. In other words, the method for detection of the present disclosure is advantageous in its ability to simply and accurately detect atopic dermatitis while reducing the burden on the patient. The method for detection of the present disclosure can also be used to perform detection based on a biological sample collected by a noninvasive method such as urine collection, and thus is advantageous also in that it can be applied to subjects for which it is difficult to detect atopic dermatitis by an invasive method such as blood collection, for example, subjects for which it is not easy to collect blood, such as children (including babies and infants).

According to another aspect of the present disclosure, a method for diagnosing atopic dermatitis is provided. The method for diagnosis according to the present disclosure can be used to diagnose whether a subject suffers from atopic dermatitis using a lipid metabolite in a biological sample as an index. In the method for diagnosis according to the present disclosure, a step (A′) of measuring the concentration of a lipid metabolite in a biological sample of a test subject is carried out in a similar manner as in the method for detection of the present disclosure. The method for diagnosis of the present disclosure may further comprise a step (B′) of comparing the concentration of the lipid metabolite in the biological sample of the subject with the concentration of the lipid metabolite in a biological sample of a healthy subject, and may also further comprise a step (C′) of, when the concentration of the lipid metabolite in the biological sample of the subject is significantly different from the concentration of the lipid metabolite in the biological sample of the healthy subject, determining that the subject suffers from atopic dermatitis. The steps (A′), (B′) and (C′) correspond to the above steps (A), (B) and (C), respectively, and can be carried out in accordance with the descriptions concerning the method for detection of the present disclosure.

According to a second aspect of the present disclosure, a method for determining a therapeutic effect on atopic dermatitis is provided. The method for determination according to the present disclosure can be used to determine a therapeutic effect on atopic dermatitis using a lipid metabolite in a biological sample as an index.

In the method for determination according to the present disclosure, a step (D) of measuring the concentration of a lipid metabolite in a biological sample of a test subject is carried out in a similar manner as in the method for detection of the present disclosure. The concentration of the lipid metabolite can be measured in a similar manner as in the method for detection of the present disclosure.

In the method for determination of the present disclosure, the step of determining a therapeutic effect on atopic dermatitis in the subject having undergone treatment based on the concentration of the lipid metabolite measured in the step (D) can further be carried out. This step may further comprise a step (E) of comparing the concentration of the lipid metabolite in the biological sample of the subject who has undergone treatment of atopic dermatitis with the concentration of the lipid metabolite in a biological sample of a healthy subject. When the concentration of the lipid metabolite in the biological sample of the subject who has undergone treatment of atopic dermatitis is significantly different from the concentration of the lipid metabolite in the biological sample of the subject before treatment or the concentration of the lipid metabolite in a biological sample of a subject suffering from atopic dermatitis, or when the concentration of the lipid metabolite in the biological sample of the subject is not significantly different from the concentration of the lipid metabolite in the biological sample of the healthy subject, it is indicated that the treatment has a therapeutic effect. That is, the method for determination of the present disclosure may further comprise a step (F) of determining that treatment of atopic dermatitis has a therapeutic effect, when the concentration of the lipid metabolite in the biological sample of the subject having undergone the treatment is significantly different from the concentration of the lipid metabolite in the biological sample of the subject before the treatment or the concentration of the lipid metabolite in a biological sample of a subject suffering from atopic dermatitis (preferably, when it is significantly different in a direction of approaching the concentration of the lipid metabolite in the biological sample of the healthy subject), or when the concentration of the lipid metabolite in the biological sample of the subject having undergone the treatment is not significantly different from the concentration of the lipid metabolite in the biological sample of the healthy subject. The term “significantly different” in the step (F) means that the concentration of the lipid metabolite in the biological sample of the subject having undergone the treatment is either higher or lower than the concentration of the lipid metabolite in the subject before the treatment or the subject suffering from atopic dermatitis depending on the type of lipid metabolite, for example, that, when the lipid metabolite is a lipid metabolite X, the concentration of the lipid metabolite in the biological sample of the subject is lower, preferably significantly lower, than the concentration of the lipid metabolite in the biological sample of the subject before the treatment or the subject suffering from atopic dermatitis (for example, the concentration of the lipid metabolite in the biological sample of the subject is statistically significantly low as compared with the concentration of the lipid metabolite in the biological sample of the subject before the treatment or the subject suffering from atopic dermatitis, or is about 0.9 times or less, about 0.8 times or less, about 0.7 times or less, about 0.6 times or less, about 0.5 times or less, about 0.4 times or less, or about 0.3 times or less thereof), and means that, when the lipid metabolite is a lipid metabolite Y, the concentration of the lipid metabolite in the biological sample of the subject is higher, preferably significantly higher, than the concentration of the lipid metabolite in the biological sample of the subject before the treatment or the subject suffering from atopic dermatitis (for example, the concentration of the lipid metabolite in the biological sample of the subject is statistically significantly high as compared with the concentration of the lipid metabolite in the biological sample of the subject before the treatment or the subject suffering from atopic dermatitis, or is about 1.1 times or more, about 1.2 times or more, about 1.3 times or more, about 1.4 times or more, about 1.5 times or more, about 1.6 times or more, about 1.7 times or more, about 1.8 times or more, about 1.9 times or more, about 2.0 times or more, about 2.5 times or more, or about 3 times or more thereof). The term “not significantly different” in the step (F) means that, for both of the lipid metabolite X and the lipid metabolite Y, the concentration of the lipid metabolite in the biological sample of the subject is equivalent to the concentration of the lipid metabolite in the biological sample of the healthy subject (for example, the concentration of the lipid metabolite in the biological sample of the subject is not statistically significantly different as compared with the concentration of the lipid metabolite in the biological sample of the healthy subject, or is more than about 0.8 times and less than about 1.2 times, or more than about 0.9 times and less than about 1.1 times thereof). As the concentration of the lipid metabolite in the biological sample of the subject before the treatment, there can be used a value obtained by measuring the concentration of the lipid metabolite in the biological sample of the subject before the treatment. As the concentration of the lipid metabolite in the biological sample of the subject suffering from atopic dermatitis, there can be used a mean value calculated by collecting biological samples from a plurality of subjects suffering from atopic dermatitis in advance and measuring the concentrations of the lipid metabolite in the biological samples. Also, as the concentration of the lipid metabolite in the biological sample of the healthy subject, there can be used a mean value calculated by collecting biological samples from a plurality of healthy subjects in advance and measuring the concentrations of the lipid metabolite in the biological samples. The subject undergoing the treatment in the method for determination of the present disclosure is preferably a subject suffering from atopic dermatitis. In the present disclosure, the “subject suffering from atopic dermatitis” may be a subject demonstrated to have atopic dermatitis from results of other inspection methods, and can be preferably a subject diagnosed as developing atopic dermatitis by a doctor or veterinarian.

The treatment of atopic dermatitis on which a therapeutic effect can be determined by the method for determination of the present disclosure include drug therapy and proactive therapy. The drug therapy includes treatment with therapeutic agents for atopic dermatitis, and examples of such therapeutic agents include pharmaceutical products such as anti-inflammatory external drugs (external steroid drugs, tacrolimus, and non-steroidal anti-inflammatory drugs), anti-histamine drugs, cyclosporine, internal steroid drugs, Chinese herbs, and antibody drugs.

The method for determination according to the present disclosure can be used to determine the therapeutic effect in the subject having undergone treatment of atopic dermatitis, thereby verifying the effectiveness of the treatment of atopic dermatitis performed on the subject. Then, if no therapeutic effect is observed, the treatment can be immediately stopped and another treatment plan can be made. Therefore, the method for determination of the present disclosure is advantageous in its ability to suppress unnecessary medication and therefore to contribute to reductions in medical expenses and burden on patients. The method for determination of the present disclosure can also be used to perform detection based on a biological sample collected by a noninvasive method such as urine collection, and thus is advantageous also in that it can be applied to subjects for which it is difficult to determine the therapeutic effect by an invasive method such as blood collection, for example, subjects for which it is not easy to collect blood, such as children (including babies and infants) and animals (e.g., pet animals such as dogs and cats).

According to a third aspect of the present disclosure, an atopic dermatitis marker comprising a lipid metabolite and use of a lipid metabolite as an atopic dermatitis marker are provided. In the present disclosure, the term “atopic dermatitis marker” refers to a substance of which the presence and amount serve as indicators of the presence or absence of development of atopic dermatitis or the severity of its symptom, and the atopic dermatitis marker can be used as a marker for detection, identification, evaluation, etc. of atopic dermatitis. In other words, according to the present disclosure, it is possible to use the lipid metabolite as a disease identification marker for atopic dermatitis, and to use the lipid metabolite to evaluate the severity of atopic dermatitis.

Since the method for determination of the present disclosure can be used to determine the effectiveness of a therapeutic agent for atopic dermatitis, a method for screening for a therapeutic agent or relieving agent for atopic dermatitis is also provided according to the present disclosure. That is, according to a fourth aspect of the present disclosure, there is provided a method for screening for a therapeutic agent or relieving agent for atopic dermatitis, comprising a step (G) of administering a candidate for a therapeutic agent or relieving agent for atopic dermatitis to a subject; and a step (H) of measuring the concentration of a lipid metabolite in a biological sample of the subject. In the method for screening of the present disclosure, the step of determining whether the candidate for the therapeutic agent or relieving agent has a therapeutic effect based on the concentration of the lipid metabolite measured in the step (H) can further be carried out. This step may further comprise a step (I) of comparing the concentration of the lipid metabolite in the biological sample of the subject after administration of the candidate for the therapeutic agent or relieving agent with the concentration of the lipid metabolite in a biological sample of a healthy subject. In the method for screening, when the concentration of the lipid metabolite in the biological sample of the subject after administration of the candidate for the therapeutic agent or relieving agent is significantly different from the concentration of the lipid metabolite in the biological sample of the subject before administration of the candidate for the therapeutic agent or relieving agent or the concentration of the lipid metabolite in a biological sample of a subject suffering from atopic dermatitis, or when the concentration of the lipid metabolite in the biological sample of the subject after administration of the candidate for the therapeutic agent or relieving agent is not significantly different from the concentration of the lipid metabolite in the biological sample of the healthy subject, it is indicated that the candidate for the therapeutic agent or relieving agent has a therapeutic effect. That is, the method for screening of the present disclosure may further comprise a step (J) of determining that the candidate for the therapeutic agent or relieving agent has a therapeutic effect, when the concentration of the lipid metabolite in the biological sample of the subject after administration of the candidate for the therapeutic agent or relieving agent is significantly different from the concentration of the lipid metabolite in the biological sample of the subject before administration of the candidate for the therapeutic agent or relieving agent or the concentration of the lipid metabolite in a biological sample of a subject suffering from atopic dermatitis (preferably, when it is significantly different in a direction of approaching the concentration of the lipid metabolite in the biological sample of the healthy subject), or when the concentration of the lipid metabolite in the biological sample of the subject after administration of the candidate for the therapeutic agent or relieving agent is not significantly different from the concentration of the lipid metabolite in the biological sample of the healthy subject, thereby making it possible to select the candidate having a therapeutic effect. The term “significantly different” in the step (J) means that the concentration of the lipid metabolite in the biological sample of the subject after administration of the candidate for the therapeutic agent or relieving agent is either higher or lower than the concentration of the lipid metabolite in the subject before administration of the candidate for the therapeutic agent or relieving agent or the subject suffering from atopic dermatitis depending on the type of lipid metabolite, for example, that, when the lipid metabolite is a lipid metabolite X, the concentration of the lipid metabolite in the biological sample of the subject is lower, preferably significantly lower, than the concentration of the lipid metabolite in the biological sample of the subject before administration of the candidate for the therapeutic agent or relieving agent or the subject suffering from atopic dermatitis (for example, the concentration of the lipid metabolite in the biological sample of the subject is statistically significantly low as compared with the concentration of the lipid metabolite in the biological sample of the subject before administration of the candidate for the therapeutic agent or relieving agent or the subject suffering from atopic dermatitis, or is about 0.9 times or less, about 0.8 times or less, about 0.7 times or less, about 0.6 times or less, about 0.5 times or less, about 0.4 times or less, or about 0.3 times or less thereof), and means that, when the lipid metabolite is a lipid metabolite Y, the concentration of the lipid metabolite in the biological sample of the subject is higher, preferably significantly higher, than the concentration of the lipid metabolite in the biological sample of the subject before administration of the candidate for the therapeutic agent or relieving agent or the subject suffering from atopic dermatitis (for example, the concentration of the lipid metabolite in the biological sample of the subject is statistically significantly high as compared with the concentration of the lipid metabolite in the biological sample of the subject before administration of the candidate for the therapeutic agent or relieving agent or the subject suffering from atopic dermatitis, or is about 1.1 times or more, about 1.2 times or more, about 1.3 times or more, about 1.4 times or more, about 1.5 times or more, about 1.6 times or more, about 1.7 times or more, about 1.8 times or more, about 1.9 times or more, about 2.0 times or more, about 2.5 times or more, or about 3 times or more thereof). The term “not significantly different” in the step (J) means that, for both of the lipid metabolite X and the lipid metabolite Y, the concentration of the lipid metabolite in the biological sample of the subject is equivalent to the concentration of the lipid metabolite in the biological sample of the healthy subject (for example, the concentration of the lipid metabolite in the biological sample of the subject is not statistically significantly different as compared with the concentration of the lipid metabolite in the biological sample of the healthy subject, or is more than about 0.8 times and less than about 1.2 times, or more than about 0.9 times and less than about 1.1 times thereof). The method for screening of the present disclosure can be carried out in accordance with the descriptions concerning the method for detection and method for determination according to the present disclosure. The subject to which the candidate for the therapeutic agent or relieving agent is administered in the method for screening of the present disclosure is preferably a subject suffering from atopic dermatitis. When the method for screening of the present disclosure is carried out, a mammal other than a human can be used as the subject. The therapeutic agent for atopic dermatitis, which is the target for the method for screening of the present disclosure, is the same as that described for the method for determination of the present disclosure. The relieving agent for atopic dermatitis, which is the target for the method for screening of the present disclosure, includes foods having a function of relieving the symptoms of atopic dermatitis (e.g., food compositions), quasi-drugs (e.g., medicated cosmetics), feeds (e.g., pet foods), cosmetics (e.g., cosmetic compositions), and skin care compositions, and the foods include supplements, food for specified health use, and foods with functional claims. In addition, the term “relieving” is used with the meaning including improvement.

According to a fifth aspect of the present disclosure, there is provided a method for identifying an atopic dermatitis marker in a lipid metabolite in a biological sample, comprising a step (K) of measuring the concentration of a lipid metabolite in a biological sample of a subject suffering from atopic dermatitis and the concentration of the lipid metabolite in a biological sample of a healthy subject; and a step (L) of comparing the measured two concentrations. In the method for identification of the present disclosure, the step of determining the lipid metabolite as an atopic dermatitis marker based on the results of comparison in concentration performed in the step (L). In this step, when the concentration of the lipid metabolite in the biological sample of the subject suffering from atopic dermatitis is significantly different from the concentration of the lipid metabolite in the biological sample of the healthy subject, it is indicated that the lipid metabolite is an atopic dermatitis marker. That is, the method for identification of the present disclosure may further comprise a step (M) of determining that the lipid metabolite is an atopic dermatitis marker when the concentration of the lipid metabolite in the biological sample of the subject suffering from atopic dermatitis is significantly different from the concentration of the lipid metabolite in the biological sample of the healthy subject. The term “significantly different” in the step (M) means that the concentration of the lipid metabolite in the subject suffering from atopic dermatitis is either higher or lower than the concentration of the lipid metabolite in the healthy subject depending on the type of lipid metabolite, for example, that, when the lipid metabolite is a lipid metabolite X, the concentration of the lipid metabolite in the biological sample of the subject is higher, preferably significantly higher, than the concentration of the lipid metabolite in the biological sample of the healthy subject (for example, the concentration of the lipid metabolite in the biological sample of the subject is statistically significantly high as compared with the concentration of the lipid metabolite in the biological sample of the healthy subject, or is about 1.1 times or more, about 1.2 times or more, about 1.3 times or more, about 1.4 times or more, about 1.5 times or more, about 1.6 times or more, about 1.7 times or more, about 1.8 times or more, about 1.9 times or more, about 2.0 times or more, about 2.5 times or more, or about 3 times or more thereof), and means that, when the lipid metabolite is a lipid metabolite Y, the concentration of the lipid metabolite in the biological sample of the subject is lower, preferably significantly lower, than the concentration of the lipid metabolite in the biological sample of the healthy subject (for example, the concentration of the lipid metabolite in the biological sample of the subject is statistically significantly low as compared with the concentration of the lipid metabolite in the biological sample of the healthy subject, or is about 0.9 times or less, about 0.8 times or less, about 0.7 times or less, about 0.6 times or less, about 0.5 times or less, about 0.4 times or less, or about 0.3 times or less thereof). The method for identification of the present disclosure may be carried out in accordance with the descriptions concerning the method for detection and method for determination according to the present disclosure.

Treatment of atopic dermatitis can be performed on the subject in which atopic dermatitis has been detected by the method for detection of the present disclosure or the subject who has been diagnosed as having atopic dermatitis by the method for diagnosis of the present disclosure. Thus, according to a sixth aspect of the present disclosure, there is provided a method for treating atopic dermatitis, comprising the step (A) of measuring the concentration of a lipid metabolite in a biological sample of a subject; the step (B) of comparing the concentration of the lipid metabolite in the biological sample of the subject with the concentration of the lipid metabolite in a biological sample of a healthy subject; the step (C) of determining that the subject suffers from atopic dermatitis when the concentration of the lipid metabolite in the biological sample of the subject is significantly different from the concentration of the lipid metabolite in the biological sample of the healthy subject; and the step (N) of performing treatment of atopic dermatitis on the subject determined to suffer from atopic dermatitis. The steps of detecting and determining atopic dermatitis (i.e., steps (A), (B) and (C)) in the method for treatment of the present disclosure can be carried out in accordance with the descriptions concerning the method for detection according to the present disclosure and the method for diagnosis of the present disclosure. The treatment of atopic dermatitis can be performed in accordance with the descriptions concerning the method for determination of the present disclosure.

According to a seventh aspect of the present disclosure, a kit for detecting atopic dermatitis, comprising a means for quantifying a lipid metabolite in a biological sample of a subject is provided. The kit of the present disclosure is typically a kit for detecting atopic dermatitis according to the method for detection of the present disclosure. Examples of the means for quantifying a lipid metabolite include a substance that specifically binds to the lipid metabolite, and the means for quantification is typically an antibody against the lipid metabolite. As the means for quantifying a lipid metabolite, a mass spectrometer for use in the mass spectrometry as described above is also indicated.

When the means for quantifying a lipid metabolite is an antibody in the kit of the present disclosure, the kit of the present disclosure comprises a reagent (and a device in some cases) necessary for measuring the concentration of the lipid metabolite by immunoassay utilizing the antibody.

Examples of the kit of the present disclosure include a kit that measures the concentration of the lipid metabolite by a sandwich method. The kit may comprise a microtiter plate, an anti-lipid metabolite antibody for capture, an anti-lipid metabolite antibody labeled with alkaline phosphatase or peroxidase, and an alkaline phosphatase substrate or peroxidase substrate.

Examples of the kit of the present disclosure also include a kit that measures the concentration of the lipid metabolite by a sandwich method using a secondary antibody. The kit may comprise a microtiter plate, an anti-lipid metabolite antibody for capture, an anti-lipid metabolite antibody as a primary antibody, an antibody against an anti-lipid metabolite antibody labeled with alkaline phosphatase or peroxidase as a secondary antibody, and an alkaline phosphatase substrate or peroxidase substrate.

The kit described above can be used, for example, in the following manner. First, the antibody for capture is fixed on the microtiter plate, and a biological sample of a subject is appropriately diluted and added thereto, and then incubated. The sample is removed and washed. Subsequently, the primary antibody is added, and incubation and washing are performed. The enzyme-labeled secondary antibody is further added, and incubation is performed. Thereafter, the substrate is added for color development. The color development can be measured using a microtiter plate reader or the like to determine the concentration of the lipid metabolite.

In the kit of the present disclosure, the labeled antibody is not limited to the enzyme-labeled antibody, and may be an antibody labeled with a radioactive substance (such as 25I, 131I, 35S or 3H), a fluorescent substance (such as fluorescein isothiocyanate, rhodamine, dansyl chloride, phycoerythrin, tetramethylrhodamine isothiocyanate, or near-infrared fluorescent material), a luminescent substance (such as luciferase, luciferin or equolin), a nanoparticle (such as colloidal gold or quantum dot), or the like. It is also possible to use a biotinylated antibody as the labeled antibody and to add labeled avidin or streptavidin to the kit.

Further examples of the kit of the present disclosure include a kit that measures the concentration of a lipid metabolite by an immunochromatography method. The kit can have a structure in which an antibody storage part in which a first anti-lipid metabolite antibody labeled with colloidal gold or the like is stored and a determination part in which a second anti-lipid metabolite antibody (preferably, an antibody that recognizes another epitope of the lipid metabolite) is fixed in a line on a cellulose membrane or the like are connected by a narrow groove.

The kit described above can be used, for example, in the following manner. First, when a biological sample is added to the antibody storage part or a biological sample receiving part adjacent to the antibody storage part, the labeled antibody and the lipid metabolite are bound in the antibody storage part to form a lipid metabolite-labeled antibody complex. The complex moves to the determination part through the groove due to a capillary phenomenon. Subsequently, when the complex is captured by the fixed second anti-lipid metabolite antibody, a red line appears in the determination part due to the plasmon effect of colloidal gold, so that the presence of the lipid metabolite can be detected. The kit, in this case, can be provided in the form of a stick, such as a stick for inspection, and the stick for inspection may be further equipped with an absorbent paper for absorbing a biological sample, a desiccant and the like.

When the means for quantifying a lipid metabolite is a mass spectrometer in the kit of the present disclosure, the kit of the present disclosure comprises an internal standard device, in some cases, in addition to the mass spectrometer. An internal standard can be used to correct the extraction efficiency and ionization efficiency for each analysis at the time of measurement with a mass spectrometer. The internal standard used in mass spectrometry includes deuterated lipid metabolites.

The kit of the present disclosure can be performed in accordance with the descriptions concerning the method for detection and method for determination according to the present disclosure, in addition to the above description.

Hereinafter, the present disclosure will be described in more detail by way of the following examples, but is not limited thereto.

### Example 1: Preparation of Atopic Dermatitis Model Mice

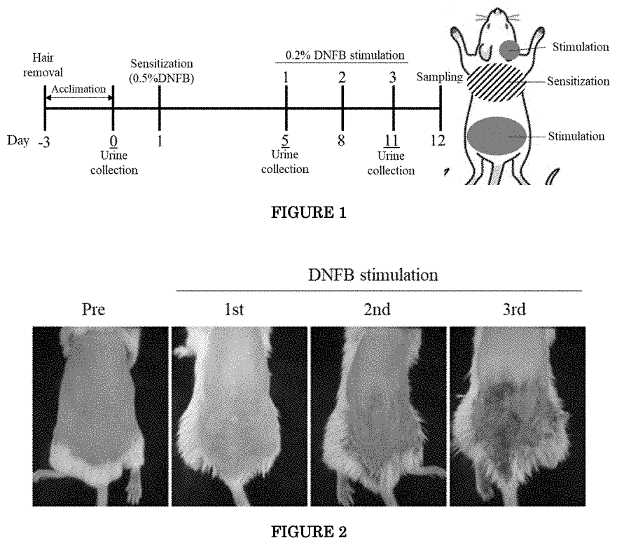

The procedures are as shown in FIG. 1. Hair on the back of BALB/C mice (male, 7 to 8 weeks old) (CLEA Japan, Inc.) was removed using hair clippers and depilatory cream under anesthesia, and the mice were acclimated for 3 days. Then, 25 μL of a 0.5% DNFB (2,4-dinitrofluorobenzene, Nacalai Tesque, Inc.) mixed solution (acetone (FUJIFILM Wako Pure Chemical Corporation): olive (FUJIFILM Wako Pure Chemical Corporation)=4:1) was dropped onto the upper back of the mice for sensitization (Day 1). Four (4) days later, 20 μL of a 0.2% DNFB mixed solution (acetone:olive=4:1) was dropped onto the right ear of the mice and 100 μL thereof was dropped onto the lower back, and first stimulation was performed. This stimulation operation was repeated every 3 days a total of 3 times (first: Day 5, second: Day 8, and third: Day 11) to prepare atopic dermatitis (AD) model mice.

### Example 2: Evaluation of Symptoms of Atopic Dermatitis

Symptoms of the AD model mice prepared in Example 1 were evaluated.

(1) Observation of Skin Pathological Condition

The change in skin pathological condition of the AD model mice over time was as shown in FIG. 2. Confirmed was a state where the inflammatory state of the back of the AD model mice worsened over a period from the first stimulation to the third stimulation.

(2) Pathological Condition Score

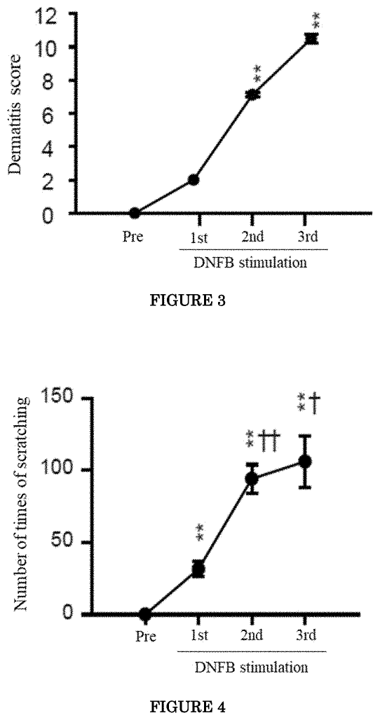

The results were as shown in FIG. 3. The pathological condition scores of a skin lesion on the back of the AD model mice on the DNFB pretreatment, the first, the second and the third DNFB stimulation were calculated based on SCORAD (Severity Scoring of Atopic Dermatitis), which is a diagnostic index of human AD. SCORAD is composed of indexes of erythema, bleeding, edema, papules, epidermolysis, erosion, scratches, dryness, lichenification and the like. Scores (0: none, 1: mild, 2: moderate, and 3: severe) are calculated according to the degree of each symptom, and the maximum score is 12 points. It was confirmed that the scores on the second and third DNFB stimulations significantly increased as compared with that of the DNFB pretreatment.

(3) Number of Times of Scratching

The results were as shown in FIG. 4. The number of times of scratching of the AD model mice was visually counted for 10 minutes on the DNFB pretreatment and 1 hour after each of the first, second, and third DNFB stimulations. Scratching with their hind leg was defined as the scratching. The scores on all the first, second and third stimulations were confirmed to significantly increase as compared with that of the DNFB pretreatment.

(4) Ear Thickness

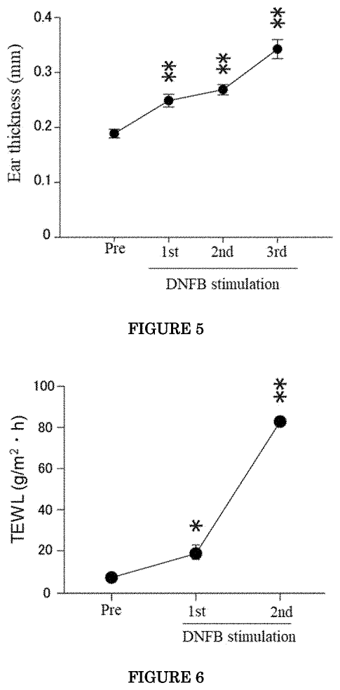

The results were as shown in FIG. 5. The ear thickness of the AD model mice was measured using an ABS solar digimatic caliper (Mitutoyo Corporation), and values in graph were calculated from a difference in ear thickness between the DNFB pretreatment and 24 hours after each stimulation.

(5) Transepidermal Water Loss (TEWL g/(m²·h))

The results were as shown in FIG. 6. The transepidermal water loss from the back of the AD model mice was measured using a multi-display device MDD4 (Courage+Khazaka).

(6) Statistical Analysis

In Examples 2 to 7, measured values were expressed as mean value±standard deviation, and all experiments were performed at least 3 times. Statistical analysis was performed using BellCurve software (Social Survey Research Information Co., Ltd.) for Excel 2015. In a significance test, two-group comparison was performed through Student's t test or Mann-Whitney U test. In addition, multi-group comparison was performed using a combination of one-way analysis of variance (ANOVA) and Tukey's test or using a combination of Kruskal-Wallis test and Steel-Dwass test. Statistical significance was defined as *P<0.05 or **P<0.01.

### Example 3: Histopathological Evaluation of Atopic Dermatitis

The skin lesion of the AD model mice prepared in Example 1 was histopathologically evaluated.

(1) Procedures

An excised skin lesion was fixed with 4% paraformaldehyde for 24 hours and embedded in paraffin to prepare a tissue section having a thickness of 4 μm. The tissue section was stained with hematoxylin-eosin (HE), chloroacetate-esterase (CAE), or May-Grünwald Giemsa (MGG) according to a conventional method. Specific staining procedures are as will be described below. The stained skin lesion tissue was observed and photographed using a BZ-X710 microscope (KEYENCE CORPORATION). The numbers of CAE-positive mast cells, neutrophils and MGG-positive eosinophils were counted within 10 randomly selected compartments per visual field (magnification: x400).

(2) Chloroacetate-Esterase (CAE) Staining

CAE staining is a staining method in which mast cell- or neutrophil-specific esterase is stained red using naphthol AS-D chloroacetate as a substrate. The tissue section specimen prepared in the above item (1) was deparaffinized and used for staining. A 4% sodium nitrite solution and a new fuchsin solution were mixed at a ratio of 1:1, and then this solution and a naphthol solution were mixed at a ratio of 1:9. This solution and a phosphate buffer (0.2 M NaH₂PO₄, 0.2 M Na₂HPO₄, pH 7.6) were mixed at a ratio of 1:20, and the section was immersed for 10 minutes. The section was washed with distilled water and then counterstained with a hematoxylin solution. The numbers of mast cells and neutrophils present within 1 mm² were counted to calculate mean values and standard deviations.

(3) May-Grünwald Giemsa (MGG) Staining

May-Grünwald Giemsa staining stains eosinophil-specific eosinophilic granules red. The tissue section specimen prepared in the above item (1) was deparaffinized and then immersed in a May-Grünwald staining solution for 6 minutes. Then, the section was washed with a 1/15 M-phosphate buffer (×10) (0.2 M NaH₂PO₄, 0.2 M Na₂HPO₄, pH 6.4 to 6.8). Thereafter, the section was immersed for 5 minutes in a solution obtained by mixing a Giemsa staining solution and distilled water at a ratio of 1:20. The number of eosinophils present within 1 mm² was counted to calculate a mean value and a standard deviation.

(4) Results

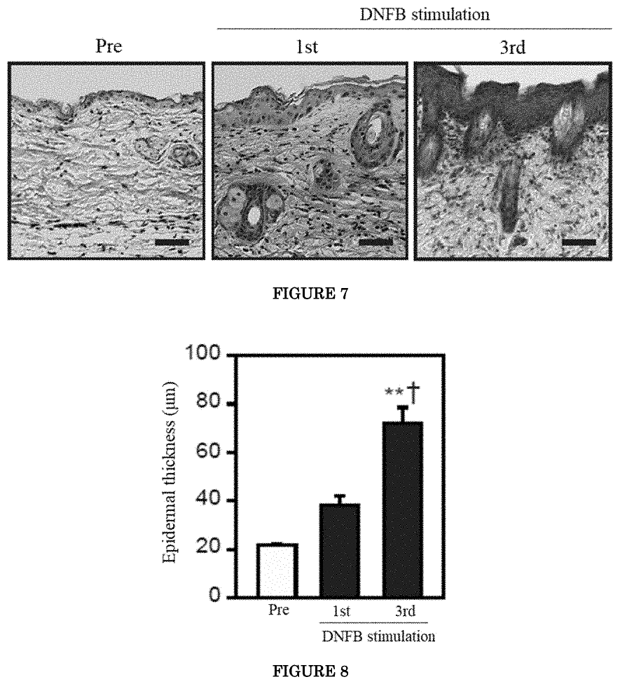

The results of the histopathological evaluations in the above items (2) and (3), which were performed on the AD model mice prepared in Example 1, were as shown in FIGS. 7 to 10.

The HE-stained images were as shown in FIG. 7. The change in epidermal thickness measured from the HE-stained images was as shown in FIG. 8 (the data is indicated as mean±standard error (n=5 to 7)).

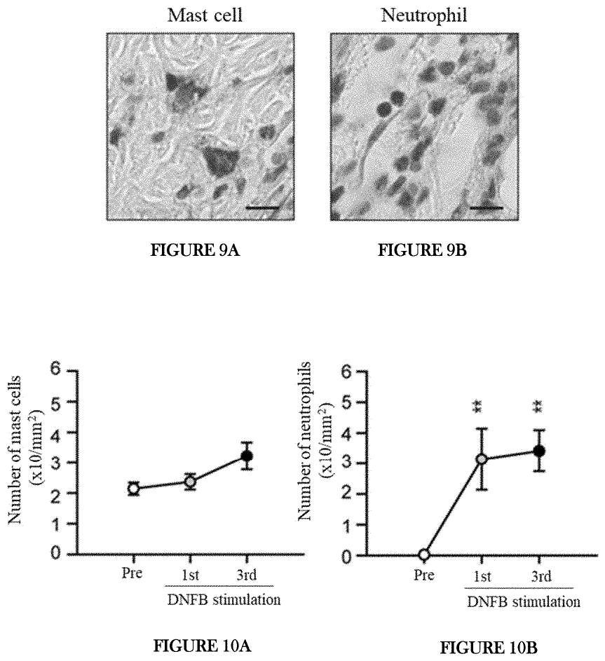

The CAE-stained images were as shown in FIGS. 9A and 9B (A: mast cells, and B: neutrophils). The changes in number of mast cells and number of neutrophils quantified from the CAE-stained images were as shown in FIGS. 10A and 10B (the data is indicated as mean±standard error (n=5 to 7)).

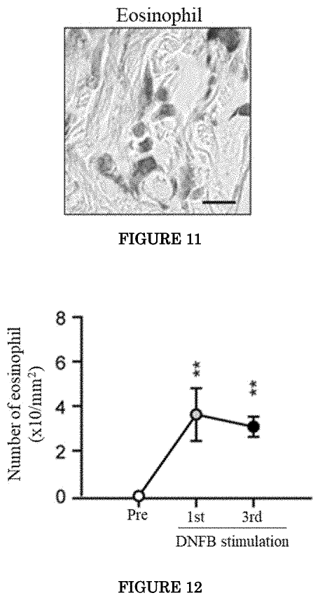

The MGG-stained images were as shown in FIG. 11. The change in number of eosinophils quantified from the MGG-stained images was as shown in FIG. 12 (the data is indicated as mean±standard error (n=5 to 7)).

From the results of Examples 2 and 3, it was confirmed that the atopic dermatitis model mice in Example 1 exhibited the symptoms of atopic dermatitis.

### Example 4: Analysis of Lipid Mediator in Urine of AD Model Mice

Lipid mediators in urine of the AD model mice (n=8) prepared in Example 1 were analyzed.

(1) Method

A. Collection of Urine

As for urine collection from the AD model mice, urine for 24 hours was collected on each of the days on the DNFB pretreatment (Day 0), the first (Day 5) and the third DNFB stimulation (Day 11) (see Table 1). The urine samples were stored at −80° C. until analysis.

B. Processing of Urine

The urine prepared in the above item A was centrifuged at 15000 rpm and 4° C. for 10 minutes. A sample solution was prepared by adding 300 μL of 0.1% formic acid water and 10 μL of an internal standard solution as indicated in Table 2 to 200 μL of the supernatant. This sample solution was loaded on a solid-phase extraction cartridge (OASIS HLB μElute, Waters), and the cartridge was washed with 200 μL of distilled water and 200 μL of hexane. Then, the lipid mediators attached to the cartridge were eluted with 100 μL of 100% methanol.

C. Measurement of Lipid Mediator

The eluate prepared in the above item B was injected into LCMS-8060, and the lipid mediators were measured by the following procedures.

<LC Conditions>

The conditions used in liquid chromatography-mass spectrometry are as follows.

Analytical column: Kinetex C8 (2.1 mm I.D.×150 mm, 2.6 μm, Phenomenex)

Mobile phase A: 0.1% formic acid

Mobile phase B: acetonitrile

Flow rate: 0.4 mL/min

Injection volume: 5 μL

Column temperature: 40° C.

Gradient program: as indicated in Table 1

TABLE 1
Gradient program

| Step | Time (min.sec) | Mobile phase A (%) | Mobile phase B (%) |
|---|---|---|---|
| 0 | 0.00 | 0 | 0 |
| 1 | 5.00 | 75 | 25 |
| 2 | 10.00 | 65 | 35 |
| 3 | 20.00 | 25 | 75 |
| 4 | 20.10 | 5 | 95 |
| 5 | 25.00 | 5 | 95 |
| 6 | 25.10 | 90 | 10 |
| 7 | 30.00 | stopped | stopped |

<MS Conditions>

Mass Spectrometer: LCMS-8060 (Shimadzu Corporation)

Measurement program: LC/MS/MS Method Package for Lipid Mediators Ver. 2

(Shimadzu Corporation)

Nebulizer gas flow rate: 3 L/min

Heating gas flow rate: 10 L/min

Interface temperature: 300° C.

Drying gas flow rate: 10 L/min

DL temperature: 250° C.

Heat block temperature: 400° C.

Ionization mode: ESI+/−

Internal standard solution: Their composition was as indicated in Table 2.

TABLE 2
Lipid mediators corresponding to compositions
of internal standard solutions

| | Concentration | |
|---|---|---|
| Name of | (ng/ml) in | |
| substance | ethanol | Corresponding lipid mediator |
| tetranor- | 25 | tetranor-PGDM, tetranor-PGEM, |
| PGEM-d6 | | tetranor-PGFM |
| 6-keto | 25 | 20-hydroxy-PGE2, |
| PGF1α-d4 | | 13,14-dihydro-15-keto-tetranor-PGF1β, |
| | | 13,14-dihydro-15-keto-tetranor-PGF1α, |
| | | 6-keto-PGF1α, |
| | | 13,14-dihydro-15-keto-tetranor-PGE2, |
| | | 6,15-diketo-13,14-dihydro-PGF1α |
| TXB2-d4 | 25 | TXB2 |
| PGF2α-d4 | 25 | iPF2α-IV, PGD3, |
| PGE2-d4 | 25 | 15-keto-PGF2α |
| PGD2-d4 | 25 | PGK2, |
| | | 11-beta-13,14-dihydro-15-keto-PGF2α, |
| | | 15-keto-PGE2, |
| | | 13,14-dihydro-15-keto-PGE2 |
| LTC4-d5 | 25 | — |
| LTB4-d4 | 25 | 10,17-DiHDoHE, |
| | | 13,14-dihydro-15-keto PGJ2 |
| 15-HETE-d8 | 25 | 17-HETE |
| 12-HETE-d8 | 25 | — |
| 5-HETE-d8 | 25 | 5-HETE, 4-HDoHE, 5-HpETE |
| PAF-d4 | 25 | — |
| OEA-d4 | 0.5 | — |
| EPA-d5 | 500 | EPA |
| DHA-d5 | 500 | — |
| AA-d8 | 500 | AA |

D. Data Processing

According to the above measurement program, the detected lipid mediators (158 types) were divided into 16 groups based on their physical properties, and internal standard substances were set for the respective groups (see Table 2). A quantification error or the like generated during lipid extraction was corrected by dividing the peak area value calculated from the chromatogram of each lipid mediator by the peak area value of the corresponding internal standard substance described above using LabSolutions LCMS (Version 5.65, Shimadzu Corporation). The concentration of each lipid mediator (vertical axis in FIGS. 13A-C to 19A and 19B and 21 to 30A and 30B) is indicated as a value obtained by dividing the peak area value of the lipid mediator by the peak area value of the internal standard substance, and then further dividing the value of creatinine in urine of each sample. The concentration of creatinine in urine was measured using LabAssay Creatinine (FUJIFILM Wako Pure Chemical Corporation).

(2) Results

<n-6 Fatty Acid>

A. Lipid Mediator at Downstream of COX

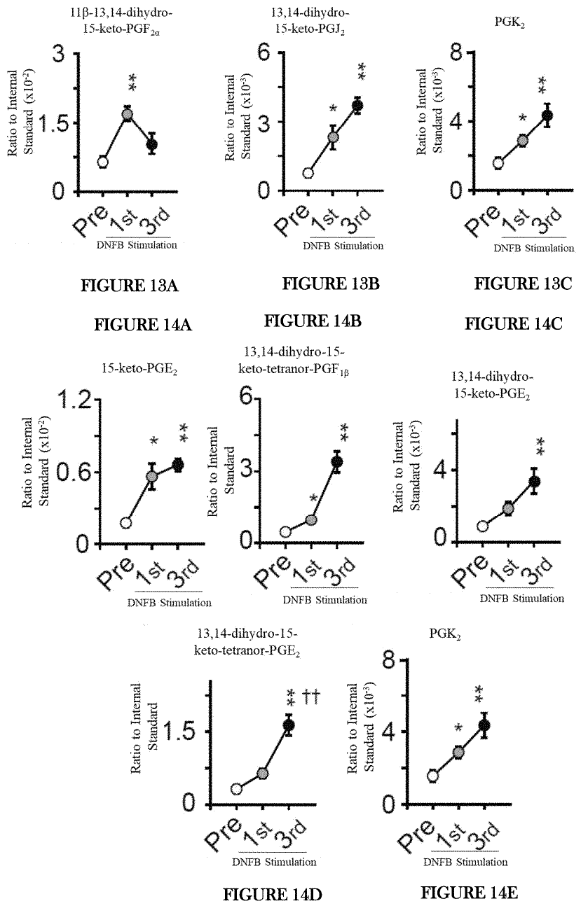

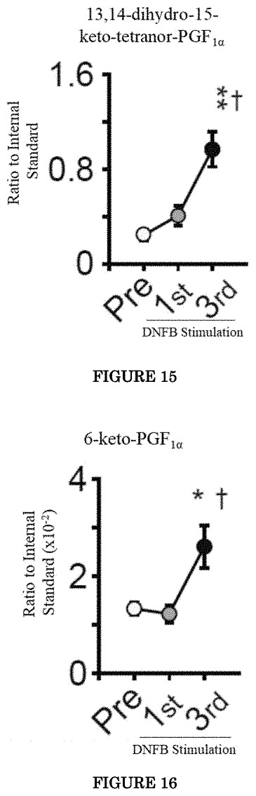

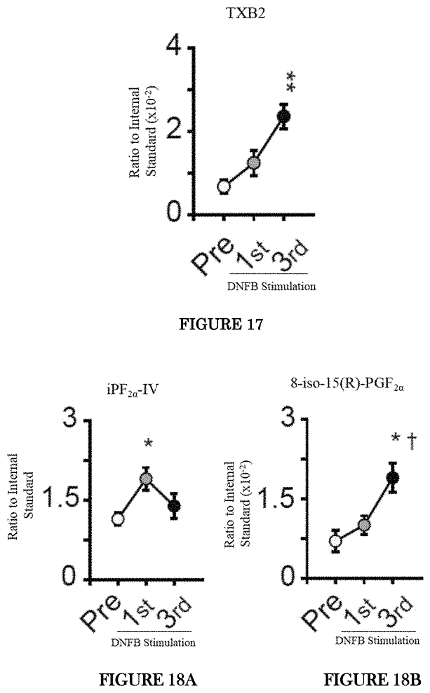

As shown in FIGS. 13A-C to 17, ten lipid mediators at the downstream of cyclooxygenase (COX) derived from arachidonic acid (sometimes abbreviated as “AA” herein) as an n-6 fatty acid were confirmed to significantly increase. Specifically, the lipid mediators metabolized from prostaglandin D2 (sometimes abbreviated as “PGD2” or “PGD₂” herein) were 11β-13,14-dihydro-15-keto-PGF2α (FIG. 13A), 13,14-dihydro-15-keto-PGJ2 (FIG. 13B), and prostaglandin K2 (sometimes abbreviated as “PGK2” or “PGK2” herein) (FIG. 13C). The lipid mediators metabolized from prostaglandin E2 (sometimes abbreviated as “PGE2” or “PGE₂” herein) were 15-keto-PGE2 (FIG. 14A), 13,14-dihydro-15-keto-tetranor-PGF1β. (FIG. 14B), 13,14-dihydro-15-keto-PGE2 (FIG. 14C), 13,14-dihydro-15-keto-tetranor-PGE2 (FIG. 14D), and PGK2 (FIG. 14E (same as FIG. 13C)). Note that PGK2 is metabolized from either PGD₂ or PGE₂. PGJ2 is prostaglandin J2 (sometimes abbreviated as “PGJ₂” herein), and PGF1β is prostaglandin F1β (sometimes abbreviated as “PGF_{1β}” herein). The lipid mediator metabolized from prostaglandin F2α (sometimes abbreviated as “PGF_{2α}” herein) was 13,14-dihydro-15-keto-tetranor-PGF1α (FIG. 15). The lipid mediator metabolized from prostaglandin I2 (sometimes abbreviated as “PGI2” herein) was 6-keto-PGF1α (FIG. 16). Note that PGF1α is prostaglandin F1α (sometimes abbreviated as “PGF_{1α}” herein). Thromboxane B2 (sometimes abbreviated as “TXB₂” herein) (FIG. 17) was also an AA-derived lipid mediator at the downstream of COX. The metabolic pathways (derived from AA) of the lipid mediators at the downstream of COX are as indicated in Table 3.

TABLE 3
Metabolic pathway of lipid mediator at downstream of COX (derived from AA)
AA
COX ↓
PGH2

| | | | | | |
|---|---|---|---|---|---|
| | | | | | TXB2 |
| PGD2 | PGD2/PGE2 | PGE2 | PGF2α | PGI2 | (FIG. 17) |

| | | | | | | |
|---|---|---|---|---|---|---|
| 11β-13,14- | 13,14- | PGK2 | 15-keto- | 13,14- | 13,14- | 6-keto- |
| dihydro- | dihydro- | (FIG. 13C, | PGE2 | dihydro- | dihydro- | PGF1α |
| 15-keto- | 15-keto- | 14E) | (FIG. 14A) | 15-keto- | 15-keto- | (FIG. 16) |
| PGF2α | PGJ2 | | | tetranor- | tetranor- | |
| (FIG. 13A) | (FIG. 13B) | | | PGF1β | PGF1α | |
| | | | | (FIG. 14B) | (FIG. 15) | |
| | | | 13,14- | | | |
| | | | dihydro- | | | |
| | | | 15-keto- | | | |
| | | | PGE2 | | | |
| | | | (FIG. 14C) | | | |
| | | | 13,14- | | | |
| | | | dihydro- | | | |
| | | | 15-keto- | | | |
| | | | tetranor- | | | |
| | | | PGE2 | | | |
| | | | (FIG. 14D) | | | |

PGH2: prostaglandin H2

B. Lipid Mediator at Downstream of Ox

As shown in FIG. 16, iso-prostaglandin F2α-IV (sometimes abbreviated as “iPF2α-IV” or “iPF_{2α}-IV” herein) (FIG. 18A) and 8-iso-15(R)-PGF2α (FIG. 18B), which are lipid mediators at the downstream of enzyme-independent oxidation (OX) derived from AA, were confirmed to significantly increase.

<n-3 Fatty Acid>

C. Lipid Mediator Derived from n-3 Fatty Acid

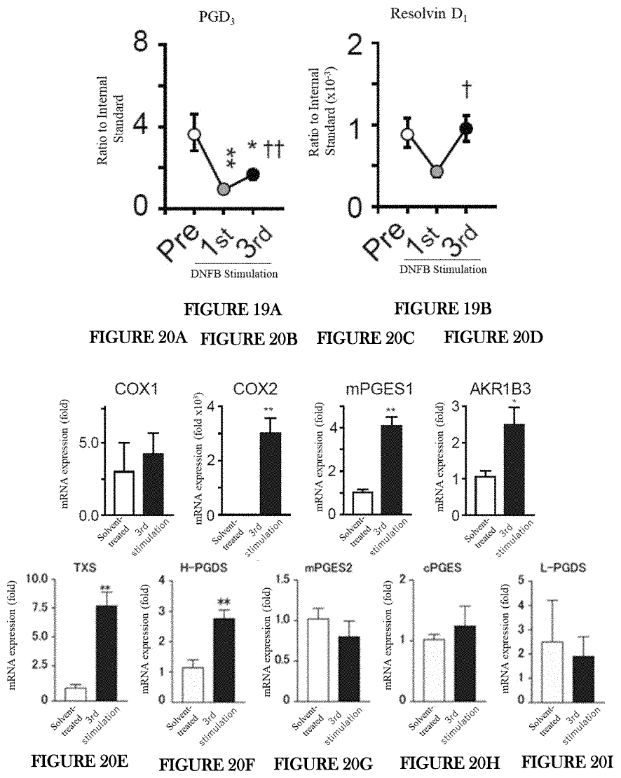

As shown in FIGS. 19A and 19B, prostaglandin D3 (sometimes abbreviated as “PGD3” or “PGD₃” herein) (FIG. 19A) and resolvin D1 (FIG. 19B), which are lipid mediators at the downstream of lipoxygenase (LOX) derived from eicosapentaenoic acid (sometimes abbreviated as “EPA” herein) and docosahexaenoic acid (sometimes abbreviated as “DHA” herein), respectively, as n-3 fatty acids, were confirmed to significantly decrease after the first stimulation.

These results suggested that the major type of fatty acid used to produce lipid mediators was n-6 fatty acid in urine of the AD model mice. Among the lipid mediators at the downstream of COX, the three PGD2-derived lipid mediators (11β-13,14-dihydro-15-keto-PGF2α, 13,14-dihydro-15-keto-PGJ2, and PGK2) (FIGS. 13A to 13C), five PGE2-derived lipid mediators (15-keto-PGE2, 13,14-dihydro-15-keto-tetranor-PGF1β, 13,14-dihydro-15-keto-PGE2, 13,14-dihydro-15-keto-tetranor-PGE2, and PGK2) (FIGS. 14A to E), one PGF2α-derived lipid mediator (13,14-dihydro-15-keto-tetranor-PGF1α) (FIG. 15), one PGI2-derived lipid mediator (6-keto-PGF1α) (FIG. 16), and TXB2 (FIG. 17) significantly increased in the DNFB-stimulated urine. Note that PGK2 (FIGS. 13C and 14E are the same figures) is metabolized from either PGD2 or PGE2.

### Example 5: Gene Expression Analysis of Lipid Metabolic Enzyme and Lipid Synthase in Skin Lesion

Gene expression analysis of lipid metabolic enzymes and lipid synthases in the skin lesion of the AD model mice prepared in Example 1 was performed.

(1) Quantitative RT-PCR

Total RNA was isolated from the skin using a trizol reagent (Molecular Research), and reverse-transcribed into cDNA using ReverTra Ace (TOYOBTO CO., LTD.). Quantitative RT-PCR was performed using THUNDERBIRD SYBR qPCR Mix (TOYOBTO CO., LTD.) and AriaMx Real-Time PCR System (Agilent Technologies) under the conditions indicated in Tables 3 to 5. Quantification was performed by the Delta Delta Ct method.

TABLE 4
Composition of reagent

| Contents | Volume | |
|---|---|---|
| SYBER Green | 5 | μL |
| Rox dye | 0.2 | μL |
| DW | 1.8 | μL |
| Primer (10 μm) | 1 μL × 2 | |
| DNA | 1 | μL |
| Total | 10 | μL |

TABLE 5
Primer sequence used in quantitative PCR

| | | SEQ |
|---|---|---|
| Gene | Sequence | ID NO: |
| Akr1B3 | Forward: GGCCGTGAAAGTTGCTATTG | 1 |
| | Reverse: ATGCTCTTGTCATGGAACGTG | 2 |
| Cox-1 | Forward: ATGAGTCGAAGGAGTCTCTCG | 3 |
| | Reverse: GCACGGATAGTAACAACAGGGA | 4 |
| Cox-2 | Forward: AAGCCGAGCACCTTTGGAG | 5 |
| | Reverse: ATTGATGGTGGCTGTTTTGGTAG | 6 |
| mpges-1 | Forward: CTAGCCGAGATGCCTTCCC | 7 |
| | Reverse: CCACCGCGTACATCTTGATG | 8 |
| mpges-2 | Forward: TCCTTGCCCTGGTCATTCAT | 9 |
| | Reverse: GGAAGGAGACAGCTTGCAAC | 10 |
| cpges | Forward: AGTTGTCTTGGAGGAAGCGA | 11 |
| | Reverse: ATGACTGGCCGGATTCTCC | 12 |
| Txs | Forward: CACAAACACGCTGTCCTTCA | 13 |
| | Reverse: TTCCCCATGAAGAGGTCCAC | 14 |
| H-pgds | Forward: TGGGAAGACAGCGTTGGAG | 15 |
| | Reverse: AGGCGAGGTGCTTGATGTG | 16 |
| L-pgds | Forward: CCTCAATCTCACCTCTACCTTCC | 17 |
| | Reverse: TCATAGTTGGCCTCCACCAC | 18 |
| 18s-rRNA | Forward: GACTCAACACGGGAAACCTCAC | 19 |
| | Reverse: CACCCACGGAATCGAGAAAG | 20 |

TABLE 6
PCR cycle condition

| | | Annealing | |
|---|---|---|---|
| Gene | Number of cycles | temperature | Product size (bp) |
| Akr1B3 | 45 | 59 | 174 |
| Cox-1 | 45 | 59 | 129 |
| Cox-2 | 45 | 59 | 147 |
| mpges-1 | 45 | 59 | 103 |
| mpges-2 | 45 | 59 | 141 |
| cpges | 45 | 59 | 142 |
| Txs | 45 | 59 | 93 |
| H-pgds | 45 | 59 | 147 |
| L-pgds | 45 | 59 | 150 |
| 18s-rRNA | 45 | 59 | 80 |

(2) Results

The results were as shown in FIGS. 20A-20I. PGD2 is produced by the activity of COX synthases (COX-1 and COX-2) and PGD synthases (H-PGDS and L-PGDS), PGE2 is produced by the activity of PGE synthases (mPGES-1, mPGES-2 and cPGES), PGF2α is produced by the activity of PGF synthase (PGFS), and TXA2 is produced by the activity of a thromboxane synthase (TXS). The aldo-keto reductase (AKR) 1B3 of the mice correlates with the PGFS activity. mRNAs of Cox-1 (FIG. 20A), Cox-2 (FIG. 20B), mPges-1 (FIG. 20C), Akr1B3 (FIG. 20D), Txs (FIG. 20E), and H-pgds (FIG. 20F) in the DNFB-treated skin 24 hours after the third stimulation significantly increased. On the other hand, the expression levels of mRNAs of mPges-2 (FIG. 20G), cPges (FIG. 20H) and L-pgds (FIG. 20I) were not affected by the DNFB treatment.

### Example 6: Immumohistochemical Analysis of Skin Lesion

Immunohistochemical analysis was performed on the skin lesion collected from the AD model mice prepared in Example 1.

(1) Immunostaining

The anatomical tissue was fixed with 4% paraformaldehyde and embedded in paraffin or an OCT compound (Sakura Finetek Japan Co., Ltd.). Sections having a thickness of 4 μm were incubated with methanol containing a 0.3% hydrogen peroxide solution at room temperature for 30 minutes. COX-1, COX-2 and mPGES-1 were stained by immersing the sections in a 50 mM Tris buffer containing 0.1% trypsin and 0.1% calcium chloride at 37° C. for 15 minutes. For staining of AKR1B3 and TBXAS1, the sections were incubated with an antigen-activating buffer (10 mM Tris, 1 mM EDTA, pH 9.0) at 95° C. for 10 minutes. After incubation with PBS containing 0.1% Triton X-100 and 5% standard goat serum at room temperature for 30 minutes, the sections were immersed in a goat anti-mPGES-1 antibody (Santa Cruz Biotechnology), a rabbit anti-TBXAS1 antibody (Abcam), a rabbit anti-AKR1B3 antibody (Osaka Bioscience Research Institute), a rabbit anti-COX-1 antibody (Cayman Chemical), and a rabbit anti-COX-2 antibody (Cayman Chemical), respectively, at a ratio of 1:200 overnight at 4° C. mPGES-1 was incubated with a biotinylated horse anti-goat antibody (VECTOR), and COX-1, COX-2, AKR1B3 and TBXAS1 were incubated with a goat anti-rabbit antibody (VECTOR) at a ratio of 1:500 for 2 hours at room temperature. After incubation with an avidin-biotin complex (VECTASTAIN) at room temperature for 30 minutes, the sections were stained by incubation with a 50 mM Tris buffer containing 200 μg/ml DAB and a 0.03% hydrogen peroxide solution. Images were captured using a BZ-X710 microscope (KEYENCE CORPORATION).

(2) Results

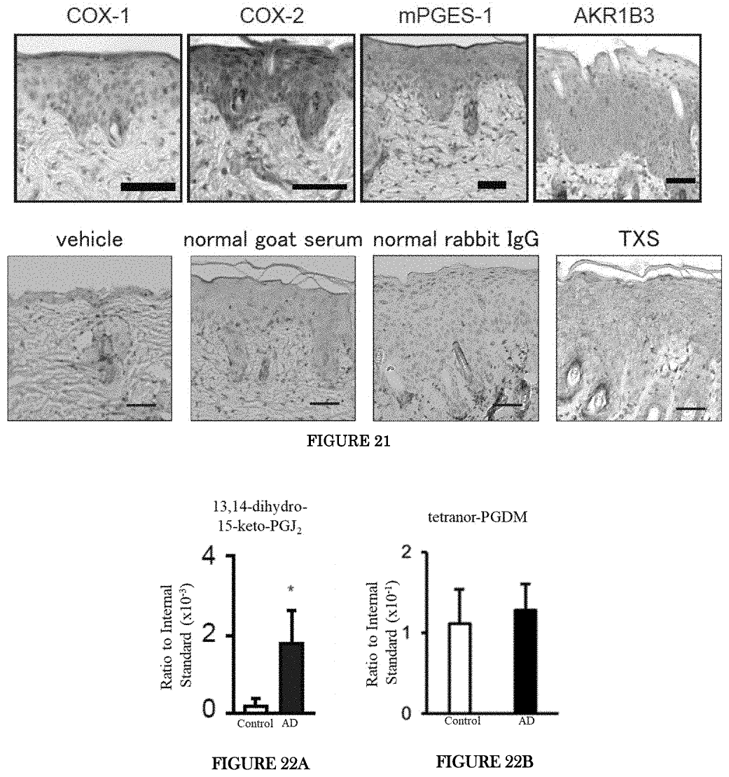

The results were as shown in FIG. 21. In immunostaining, staining of COX-2, mPGES-1, AKR1B3, and TXS was observed in the epidermal layer (FIG. 21). COX-2 and mPGES-1 were also observed in several infiltrating cells. COX-1 was weakly stained. No positive staining was observed in normal serum or a control (solvent) skin (FIG. 21). These results suggest that the lipid mediators derived from PGE2 and PGF2α are the major lipid mediators in urine of the AD model mice.

### Example 7: Analysis of Lipid Mediator in Urine of Atopic Dermatitis Patient

Analysis was performed on the lipid mediators in urine collected from an atopic dermatitis (AD) patient group (13 patients) and a control group (4 patients).

(1) Method

A. Urine Specimen

Urine specimens collected from 17 allergic patients who regularly visited the Allergy Department of the National Center for Child Health and Development were used. Among the allergic patients, 13 patients clinically diagnosed as having atopic dermatitis were classified into the AD patient group, and 4 patients having no clinical symptom (such as eczema) were classified into the control group. The clinical characteristics of the test subjects are as indicated in Table 6. The collected urine was stored at −80° C. until analysis.

TABLE 7
Clinical characteristic of test subject

| Variable | Atopic dermatitis group | Control group |
|---|---|---|
| Number of test subjects | 13 | 4 |
| Age (years old) | 8.1 ± 3.6 | 9.3 ± 2.2 |
| Sex (male, %) | 70 | 100 |
| Serum TARC (pg/mL) | 1640.4 ± 1454.4 | |
| Total serum IgE (IU/mL) | 4115.1 ± 4666.9 | |
| EASI | 13.9 ± 11.9 | |
| SCORAD | 39.0 ± 19.1 | |

The data is indicated as mean ± standard deviation.

All the test subjects agreed to informed consent, and the research protocol was approved by the Ethics Committees of the University of Tokyo and of the National Center for Child Health and Development. All experiments were performed according to the approved guidelines.

B. Processing of Urine

Urine was adjusted in a similar manner as described in Example 4, item (1), B.

C. Measurement of Lipid Mediator

The lipid mediators were measured in a similar manner as described in Example 4, item (1), C.

D. Data Processing

The data was processed in a similar manner as described in Example 4, item (1), D.

(2) Results

<n-6 Fatty Acid>

A. Lipid Mediator at Downstream of COX

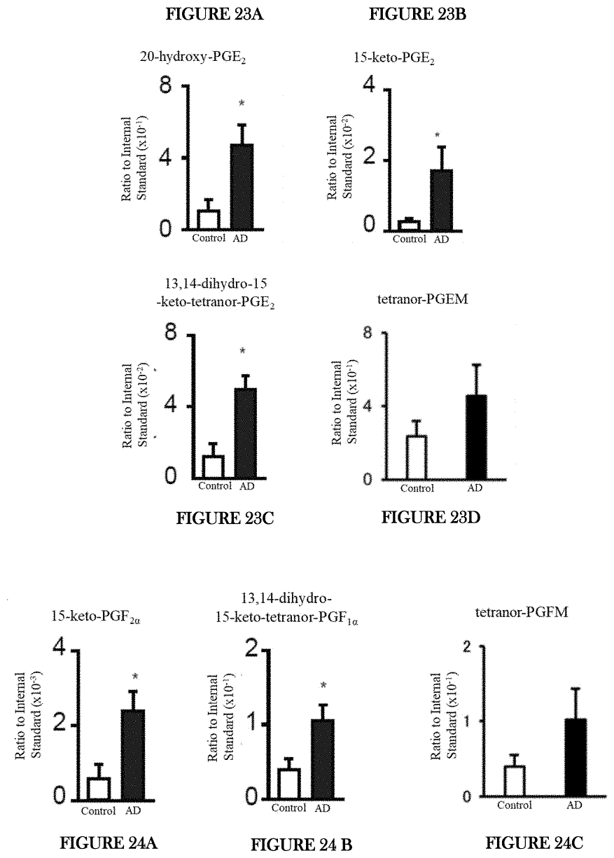

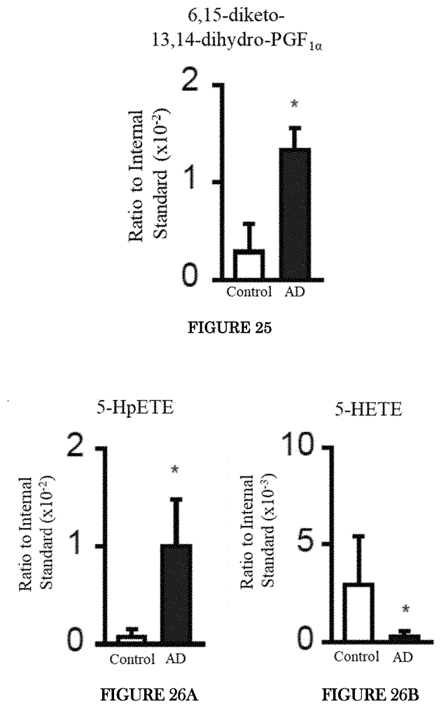

As shown in FIGS. 22A and 22B to 25, the amounts of seven AA-derived lipid mediators produced at the downstream of COX were significantly larger in the AD patient group than in the control group. Specifically, the lipid mediators metabolized from PGD2 were 13,14-dihydro-15-ketogenic-PGJ2 (FIG. 22A) and tetranor-PGDM (FIG. 22B). The lipid mediators metabolized from PGE2 were 20-hydroxy-PGE2 (FIG. 23A), 15-keto-PGE2 (FIG. 23B), 13,14-dihydro-15-keto-tetranor-PGE2 (FIG. 23C) and tetranor-PGEM (FIG. 23D). The lipid mediators metabolized from PGF2α were 15-ketogenic-PGF2α (FIG. 24A), 13,14-dihydro-15-keto-tetranor-PGF1α (FIG. 24B) and tetranor-PGFM (FIG. 24C). The lipid mediator metabolized from PGI2 was 6,15-diketo-13,14-dihydro-PGF1α (FIG. 25). The metabolic pathways (derived from AA) of the lipid mediators at the downstream of COX are as indicated in Table 8.

TABLE 8
Metabolic pathway of lipid mediator at downstream of COX (derived from AA)
AA
COX ↓
PGH2

| PGD2 | | PGE2 | | PGF2α | PGI2 |
|---|---|---|---|---|---|
| 13,14- | tetranor-PGDM | 20-hydroxy- | 15-keto-PGE2 (FIG. 23B) | 15-keto- | 6,15- |
| dihydro-15- | (FIG. 22B) | PGE2 | | PGF2α | diketo-13,14- |
| keto-PGJ2 | | (FIG. 23A) | | (FIG. 24A) | dihydro-PGF1α |
| (FIG. 22A) | | | | | (FIG. 25) |

| | | |
|---|---|---|
| 13,14- | tetranor- | 13,14- |
| dihydro-15-keto- | PGEM | dihydro-15-keto- |
| tetranor-PGE2 | (FIG. 24D) | tetranor-PGF1α |
| (FIG. 23C) | | (FIG. 24B) |
| | | tetranor- |
| | | PGFM |
| | | (FIG. 24C) |

PGH2: prostaglandin H2

B. Lipid Mediator at Downstream of LOX

As shown in FIGS. 26A and 26B, the amount of 5-hydroperoxyicosatetraenoic acid (sometimes abbreviated as “HpETE” herein) (FIG. 26A) as an AA-derived lipid mediator produced at the downstream of LOX was significantly larger in the AD patient group than that in the control group, whereas the amount of its metabolite 5-hydroxyeicosatetraenoic acid (sometimes abbreviated as “HETE” herein) (FIG. 26B) produced was significantly smaller in the AD patient group than in the control group.

C. Lipid Mediator at Downstream of CYP

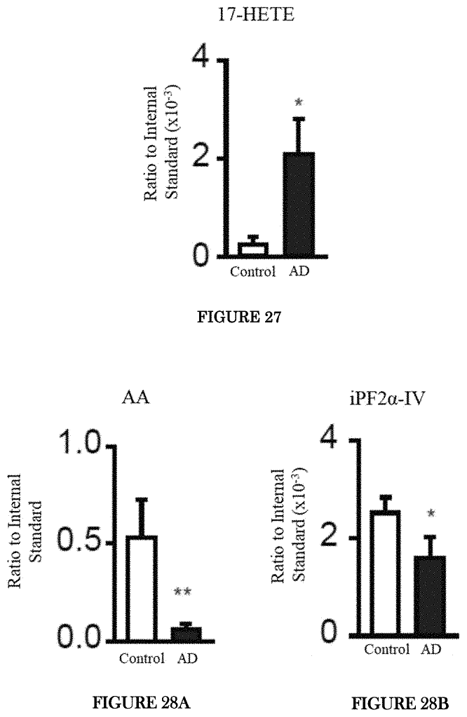

As shown in FIG. 27, the amount of 17-HETE as an AA-derived lipid mediator produced at the downstream of cytochrome p450 (CYP) was significantly larger in the AD patient group than that in the control group.

D. AA and AA-Derived Lipid Mediator at Downstream of OX

As shown in FIG. 26, the amounts of AA (FIG. 28A) and iPF2α-IV (FIG. 28B) as an AA-derived lipid mediator at the downstream of enzyme-independent oxidation (OX), were significantly smaller in the AD patient group than in the control group.

<n-3 Fatty Acid>

E. EPA and EPA-Derived Lipid Mediator

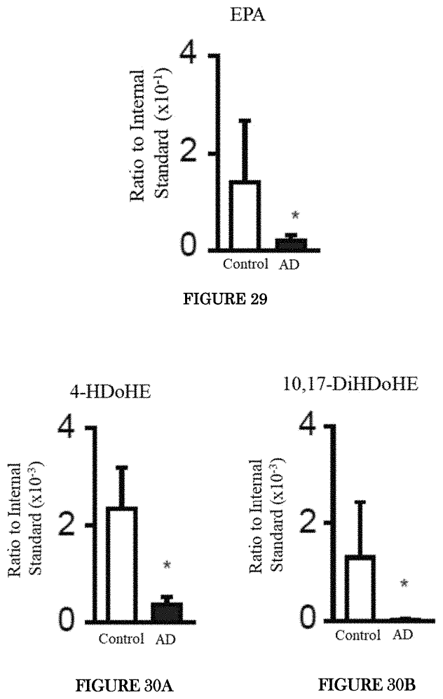

As shown in FIG. 29, the amount of EPA (FIG. 29) was significantly smaller in the AD patient group than that in the control group.

F. DHA-Derived Lipid Mediator

As shown in FIGS. 30A and 30B, the amounts of 4-hydroxy Docosahexaenoic Acid (sometimes abbreviated as “HDoHE” herein) (FIG. 30A) and 10,17-dihydroxy docosahexaenoic acid (sometimes abbreviated as “DiHDoHE” herein) (FIG. 30B) as DHA-derived lipid mediators at the downstream of LOX were significantly smaller in the AD patient group than in the control group.

### Example 8: Study with Non-Allergic Dermatitis Model Mice

In Example 8, in order to verify the specificity of lipid metabolism in AD model mice, a study was conducted using non-allergic dermatitis model mice in which non-allergic dermatitis was caused by tape stripping.

(1) Method

A. Tape Stripping

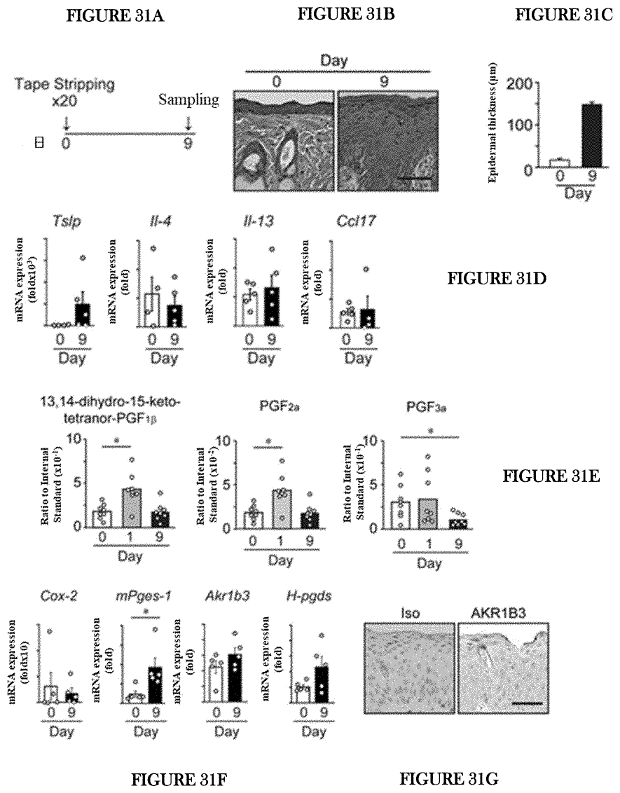

Using BALB/C mice (male, 7 to 8 weeks old) (CLEA Japan, Inc.), tape stripping was performed 20 times to cause non-allergic dermatitis (see FIG. 31A, as for the time series of the experiment).

B. HE Staining

HE staining was performed in a similar manner as described in Example 3, item (1).

C. Counting of Eosinophil, Neutrophil and Mast Cell

Counting was performed in a similar manner as described in Example 3, item (2).

D. Collection and Processing of Urine

Urine was collected and processed in a similar manner as described in Example 4, item (1), A and B, except that urine was collected on the day of tape stripping treatment (Day 0) and on Days 1 and 9 after the treatment.

E. Measurement of Lipid Mediator

Measurement was performed in a similar manner as described in Example 4, item (1), C.

F. Data Processing

The data was processed in a similar manner as described in Example 4, item (1), D.

G. Quantitative RT-PCR

Quantitative RT-PCR was performed in a similar manner as described in Example 5, item (1).

H. Immunostaining

Immunostaining was performed in a similar manner as described in Example 6, item (1).

(2) Results

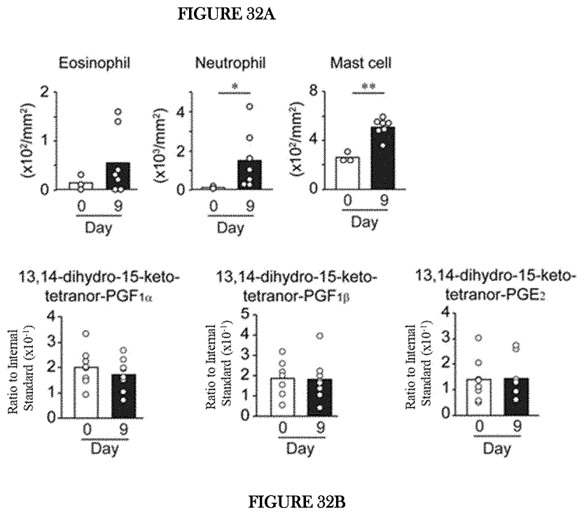

The results were as shown in FIGS. 31A-30G and 32A and 32B. From the change in epidermal thickness measured from the HE-stained images (FIG. 31B), an increase in epidermal thickness was confirmed on Day 9 after the tape stripping treatment (FIG. 31C). In the quantitative RT-PCR, it was confirmed that the expression level of mRNA of Tslp tended to increase, whereas the expression levels of mRNAs of interleukin 4 (Il-4), interleukin 13 (Il-13) and Ccl17, as cytokines mainly produced by Th2 cells, did not tend to increase (FIG. 31D). From the report that, 6 hours after the tape stripping treatment, activation of IL-1 and protease-activated receptor 2 (PAR-2) led to activation of NF-κB and increase in TSLP level in the skin (Redhu D, et al., Br J Dermatol 2020; 182: 119-29), it was considered that Th1-type inflammation mainly mediated by IL-1 is caused in the skin subjected to the tape stripping treatment (Sanmiguel J C, et al., Cell Signal 2009; 21:685-94). Nine (9) days after the tape stripping, neutrophils and mast cells infiltrated the dermis, but eosinophils did not infiltrate the dermis (FIG. 32A).

In addition, as a result of analysis of the urine on Day 1 and the urine on Day 9 after the tape stripping treatment, 13,14-dihydro-15-keto-tetranor-PGF1β and PGF2α, as PGE2 metabolites in the urine, increased on Day 1 after the tape stripping treatment (FIG. 31E, left and middle), whereas metabolites of PGF2α and PGE2 did not increase on Day 9 after the tape stripping treatment (FIG. 32B). In contrast, the level of PGF3α, as a metabolite of EPA, significantly reduced on Day 9 after the stripping treatment (FIG. 31E, right). It was also confirmed that the tape stripping treatment did not alter the mRNA expressions of Cox-2, Akr1b3 and H-pgds except mPges-1 (FIG. 31F). The tape stripping slightly increased the expression level of AKR1B3 protein (FIG. 31G), but the degree of increase was much smaller than that in the DNFB-stimulated skin.

These results demonstrate that, unlike in the case of the non-allergic dermatitis model mice, thickened keratinocytes with allergic (Th2-type) inflammation produce PGD2, PGE2, PGF2α and PGI2 in the AD model mice, and that their metabolites are excreted in urine of the AD model mice.

1. A method for diagnosis of atopic dermatitis, comprising:
measuring the concentration of a lipid metabolite in a biological sample of a subject;
comparing the concentration of the lipid metabolite in the biological sample of the subject with the concentration of the lipid metabolite in a biological sample of a healthy subject; and
indicating that the subject suffers from atopic dermatitis based on determining that the concentration of the lipid metabolite in the biological sample of the subject is significantly different from the concentration of the lipid metabolite in the biological sample of the healthy subject.

2. (canceled)

3. (canceled)

4. The method for diagnosis according to claim 1, wherein the biological sample of the subject is a bodily fluid.

5. A method for determining a therapeutic effect of a treatment on atopic dermatitis, comprising:
measuring the concentration of a lipid metabolite in a biological sample of a subject after treatment for atopic dermatitis;
comparing the concentration of the lipid metabolite in the biological sample of the subject after treatment for atopic dermatitis with the concentration of the lipid metabolite in a biological sample from the subject before treatment for atopic dermatitis or the concentration of the lipid metabolite in a biological sample of a healthy subject; and
indicating that treatment has had a therapeutic effect based on determining that the concentration of the lipid metabolite in the biological sample of the subject after treatment for atopic dermatitis is significantly different from the concentration of the lipid metabolite in a biological sample of the subject before treatment or from the concentration of the lipid metabolite in a biological sample of a subject from atopic dermatitis.

6. (canceled)

7. (canceled)

8. The method for determining according to claim 5, wherein treatment of atopic dermatitis is drug therapy or proactive therapy.

9. The method for determining according to claim 5, wherein the biological sample is a bodily fluid.

10. The method for diagnosis according to claim 1, wherein the lipid metabolite is one or more lipid metabolite(s) selected from the group consisting of an arachidonic acid metabolite, an eicosapentaenoic acid metabolite, and a docosahexaenoic acid metabolite.

11. The method for diagnosis according to claim 1, wherein the lipid metabolite tends to have a higher concentration in the biological sample of the subject suffering from atopic dermatitis than in the biological sample of the healthy subject.

12. The method for diagnosis according to claim 11, wherein the lipid metabolite is one or more lipid metabolite(s) selected from the group consisting of 13,14-dihydro-15-keto-PGJ2, tetranor-PGDM, 20-hydroxy-PGE2, 15-keto-PGE2, 13,14-dihydro-15-keto-tetranor-PGE2, tetranor-PGEM, 15-keto-PGF2α, 13,14-dihydro-15-keto-tetranor-PGF1α, tetranor-PGFM, 6,15-diketo-13,14-dihydro-PGF1α, 5-HpETE, 17-HETE, 11β-13,14-dihydro-15-keto-PGF2α, PGK2, 13,14-dihydro-15-keto-tetranor-PGF1β, 13,14-dihydro-15-keto-PGE2, 6-keto-PGF1α, and TXB2.

13. The method for diagnosis according to claim 1, wherein the lipid metabolite tends to have a lower concentration in the biological sample of the subject suffering from atopic dermatitis than in the biological sample of the healthy subject.

14. The method for diagnosis according to claim 13, wherein the lipid metabolite is one or more lipid metabolite(s) selected from the group consisting of 5-HETE, arachidonic acid (AA), 11β-13,14-dihydro-15-keto-PGF2α, iPF2α-IV, EPA, 4-HDoHE, 10,17-DiHDoHE, and PGD3.

15-18. (canceled)

19. A method for screening for a therapeutic agent or relieving agent for atopic dermatitis, comprising:
administering a candidate for a therapeutic agent or relieving agent for atopic dermatitis to a subject suffering from atopic dermatitis;
measuring the concentration of a lipid metabolite in a biological sample of the subject suffering from atopic dermatitis;
comparing the concentration of the lipid metabolite in the biological sample of the subject suffering from atopic dermatitis after administration of the candidate for the therapeutic agent or relieving agent with a concentration of the lipid metabolite in a biological sample of the subject suffering from atopic dermatitis before administration of the candidate for the therapeutic agent or relieving agent or with a concentration of the lipid metabolite in a biological sample of a healthy subject; and
indicating that the candidate for a therapeutic agent or relieving agent for atopic dermatitis has had a therapeutic effect based on determining that the concentration of the lipid metabolite in the biological sample of the subject after administration of the candidate for the therapeutic agent or relieving agent is significantly different from the concentration of the lipid metabolite in the biological sample of the subject before administration of the candidate for the therapeutic agent or relieving agent or from the concentration of the lipid metabolite in a biological sample of a subject suffering from atopic dermatitis.

20-24. (canceled)

25. A method for treating atopic dermatitis, comprising the step (A) of measuring the concentration of a lipid metabolite in a biological sample of a subject; the step (B) of comparing the concentration of the lipid metabolite in the biological sample of the subject with the concentration of the lipid metabolite in a biological sample of a healthy subject; the step (C) of determining that the subject suffers from atopic dermatitis when the concentration of the lipid metabolite in the biological sample of the subject is significantly different from the concentration of the lipid metabolite in the biological sample of the healthy subject; and the step (N) of performing treatment of atopic dermatitis on the subject determined to suffer from atopic dermatitis.

26. The method for determination according to claim 5, further comprising:
indicating that treatment has had a therapeutic effect based on determining that the concentration of the lipid metabolite in the biological sample of the subject is not significantly different from the concentration of the lipid metabolite in the biological sample of a healthy subject.

27. The method for screening according to claim 19, further comprising:
indicating that the candidate for a therapeutic agent or relieving agent for atopic dermatitis has had a therapeutic effect based on determining that the concentration of the lipid metabolite in the biological sample of the subject after administration of the candidate for the therapeutic agent or relieving agent is not significantly different from the concentration of the lipid metabolite in a biological sample of a healthy subject.

28. The method for determining according to claim 5, wherein the lipid metabolite is one or more lipid metabolite(s) selected from the group consisting of an arachidonic acid metabolite, an eicosapentaenoic acid metabolite, and a docosahexaenoic acid metabolite.

29. The method for determining according to claim 5, wherein the lipid metabolite tends to have a higher concentration in the biological sample of the subject suffering from atopic dermatitis than in the biological sample of the healthy subject.

30. The method for determining according to claim 29, wherein the lipid metabolite is one or more lipid metabolite(s) selected from the group consisting of 13,14-dihydro-15-keto-PGJ2, tetranor-PGDM, 20-hydroxy-PGE2, 15-keto-PGE2, 13,14-dihydro-15-keto-tetranor-PGE2, tetranor-PGEM, 15-keto-PGF2α, 13,14-dihydro-15-keto-tetranor-PGF1α, tetranor-PGFM, 6,15-diketo-13,14-dihydro-PGF1α, 5-HpETE, 17-HETE, 11β-13,14-dihydro keto-PGF2α, PGK2, 13,14-dihydro-15-keto-tetranor-PGF1β, 13,14-dihydro-15-keto-PGE2, 6-keto-PGF1α, and TXB2.

31. The method for determining according to claim 5, wherein the lipid metabolite tends to have a lower concentration in the biological sample of the subject suffering from atopic dermatitis than in the biological sample of the healthy subject.

32. The method for determining according to claim 31, wherein the lipid metabolite is one or more lipid metabolite(s) selected from the group consisting of 5-HETE, arachidonic acid (AA), 11β-13,14-dihydro-15-keto-PGF2α, iPF2α-IV, EPA, 4-HDoHE, 10,17-DiHDoHE, and PGD3.
